(19) United States
(12) Patent Application Publication
(10) Pub. No.: US 2019/0060404 A1
(43) Pub. Date: Feb. 28, 2019

(54) USE OF VEGF-B FOR TREATING DISEASES INVOLVING NEOANGIOGENESIS

(71) Applicant: ZHONGSHAN OPHTHALMIC CENTER, SUN YAT-SEN UNIVERSITY, Guangzhou (CN)

(72) Inventors: Xuri Li, Guangzhou (CN); Chunsik Lee, Guangzhou (CN); Xiangrong Ren, Guangzhou (CN)

(21) Appl. No.: 16/118,486

(22) Filed: Aug. 31, 2018

(30) Foreign Application Priority Data
Aug. 31, 2017 (CN) 201710776788.9

(51) Int. Cl. A61K 38/18; A61K 45/06; A61P 35/00; C07K 14/49

(52) U.S. Cl. CPC A61K 38/1866; C07K 14/49; A61P 35/00; A61K 45/06

(57) ABSTRACT

The invention discloses a method for treating a disease involving neoangiogenesis, including administering VEGF-B to a subject; and, a pharmaceutical composition containing VEGF-B protein, VEGF-B expressing plasmids, VEGF-B expressing viruses and/or VEGF-B expressing cells as active ingredients for treating a disease involving neoangiogenesis. The VEGF-B of the invention is able to bind to FGF2 receptors FGFR1 and FGFR2, induces the formation of FGFR1/VEGFR1 or FGFR2/VEGFR1 complex, inhibits the functions of FGFR1 and FGFR2, up-regulates Spry4 expression, and inhibits FGF2 from activating Erk, thus inhibiting neoangiogenesis.

### CROSS-REFERENCE TO RELATED APPLICATIONS

This present application claims the benefit of Chinese Patent Application No.: 201710776788.9 filed on Aug. 31, 2017, the contents of which are hereby incorporated by reference.

### FIELD OF THE INVENTION

The invention relates to the field of biomedical technology, particularly the application of VEGF-B in preparing medicaments for inhibiting tumor growth.

### BACKGROUND OF THE INVENTION

VEGF-B (Vascular endothelial growth factor B) belongs to VEGF family and is expressed in varieties of cells. However, few researches have been done in regards to its function in vascular system. Currently, functions and mechanisms of VEGF-B in neoangiogenesis still remain unclear. In 1996, VEGF-B was discovered, with its amino acids sequence being 47% and 37% in homology with VEGF₁₆₅ and PlGF (Placental Growth Factor), another two members of the VEGF family. VEGF-B is expressed in most tissues and organs in form of secretory homodimer. Mature VEGF-B has two subtypes: VEGF-B₁₆₇ and VEGF-B₁₈₆. VEGF-B₁₆₇ has one binding site for heparin at its carboxyl terminal, and hereby binds to heparan sulfate proteoglycans (HSPGs) after secreted. VEGF-B₁₈₆ has no binding site for heparin and thus has a relatively dispersed distribution after being secreted from cells. VEGF-B can bind to receptors VEGFR1 and NRP-1.

As a receptor for VEGF-B in varieties of cells, VEGFR1 is expressed in cells including vascular endothelial cells and smooth muscle cells. Researches on functions of VEGFR1 in blood vessel indicate a duality thereof: in a specific condition, VEGFR1 can function as promoting or inhibiting neoangiogenesis. In some research models, the knockout of VEGFR1 promotes neoangiogenesis, VEGFR1 can inhibit the activation of Erk (extracellular regulated protein kinase) in vascular cells and non-vascular cells. However, the mechanism of VEGFR1 on inhibiting the activation of Erk and neoangiogenesis remain unclear, it is yet unclear whether VEGF-B participates in such inhibition as a ligand for VEGFR1.

Fibroblast growth factor 2 (FGF2), and its receptors FGFR1 and FGFR2 are widely expressed in the organism, and have strong effect on promoting neoangiogenesis. The over-expressed FGF2 can significantly induce neoangiogenesis, while the deficiency of FGF2 causes a decreased cardiovascular density. The knockout of FGF2 can not only affects vascularization, but also causes vascular degeneration. Mutations and dysfunction of ligands or receptors in FGF/FGFR pathway can cause tumorigenesis, such as squamous cell cancer in breast, bladder, lung and head and neck, FGF/FGFR is highly expressed in varieties of tumor cells. Therefore, it is crucial to control/inhibit the functions of FGF/FGFR for inhibiting tumorigenesis. So far, little has been known about factors responsible for inhibiting the activity of FGF2 and FGFR1/2.

### SUMMARY OF THE INVENTION

The invention aims to overcome the aforesaid drawbacks of prior art as to provide a medicament capable of inhibiting the activity of FGF2 and FGFR1/2, and thus inhibiting tumorigenesis.

In order to achieve the purpose of the invention, the invention adopts the following technical scheme:

As a first aspect, the invention provides a method of treating a disease involving neoangiogenesis in a patient, comprising: administering VEGF-B to the subject. By an integrated use of various experimental models and methods in the present application, the inventor discovered for the first time that VEGF-B is an important negative regulator of the FGF2/FGFR signaling pathway; and that VEGF-B can bind to receptors FGFR1 and FGFR2 of FGF2, induce the formation of FGFR1/VEGFR1 or FGFR2/VEGFR1 complex, up-regulate the expression of Spry4 and inhibit FGF2 from activating Erk, and thus inhibiting neoangiogenesis.

Preferably, the VEGF-B is in the form of VEGF-B protein, VEGF-B expressing plasmids, VEGF-B expressing viruses and/or VEGF-B expressing cells.

Preferably, the VEGF-B is VEGF-B₁₆₇ and/or VEGF-B₁₈₆.

Preferably, the VEGF-B is a modified VEGF-B, the modified VEGF-B is a cyclized, phosphorylated and/or methylated VEGF-B; or the VEGF-B is a recombinant protein or polypeptide having 1-5 more or less amino acids than the VEGF-B.

Preferably, the concentration of the VEGF-B is 10-300 ng/ml.

Preferably, the method further comprises: administering an inhibitor of FGF2 receptor to the subject.

Preferably, the FGF2 receptor is FGFR1 and/or FGFR2.

Preferably, the disease involving neoangiogenesis is a proliferative disease; more preferably, the proliferative disease is a cancer; more preferably, the cancer is selected from the group consisting of liver cancer, endometrial cancer, breast cancer, bladder cancer, rectal cancer, cervical cancer, ovarian cancer and melanoma.

Preferably, the VEGF-B inhibits the neoangiogenesis by inhibiting an FGF2-induced phosphorylation of Erk.

Preferably, the VEGF-B inhibits the FGF2-induced phosphorylation of Erk by competing with FGF2 for binding to FGFR1 and/or FGFR2.

Preferably, the VEGF-B inhibits the FGF2-induced phosphorylation of Erk by up-regulating Spry4 expression.

Preferably the VEGF-B up-regulates the Spry4 expression by inducing the formation of an FGFR1/VEGFR1 complex and/or an FGFR2/VEGFR1 complex.

As a second aspect, the invention further provides a pharmaceutical composition for treating a disease involving neoangiogenesis, comprising VEGF-B protein, VEGF-B expressing plasmids, VEGF-B expressing viruses and/or VEGF-B expressing cells.

Preferably, the pharmaceutical composition further comprises an inhibitor of FGF2 receptor; more preferably, the FGF2 receptor is FGFR1 and/or FGFR2.

In summary, the advantages of the invention are as follows:

VEGF-B binds to FGF2 receptors FGFR1 and FGFR2, induce the formation of FGFR1/VEGFR1 or FGFR2/VEGFR1 complex, up-regulate the expression of Spry4 and inhibit FGF2 from activating Erk, and thus inhibiting neoangiogenesis, tumor growth and proliferation of other cells and tissues.

### BRIEF DESCRIPTION OF DRAWINGS

FIG. 1A-1E show the results of embodiment 1 of the invention, wherein:

FIG. 1A shows the result of an immunoblot assay on samples from HREC (Human Retinal Endothelial Cells) stimulated with VEGF-B (right) or FGF2 (left) for different time periods (0, 15 and 30 mins); the result shows pFGFR1 (phosphorylated FGFR1) and tFGFR1 (total FGFR1) level in the said HREC;

FIG. 1B shows the result of an immunoblot assay on samples from HUVSMC (Human Umbilical Vein Smooth Muscle Cells) stimulated with VEGF-B (right) or FGF2 (left) for different time periods (0, 15 and 30 mins); the result shows pFGFR1 (phosphorylated FGFR1) and tFGFR1 (total FGFR1) level in the said HUVSMC;

FIG. 1C shows the result of an SPR (Surface Plasmon Resonance) assay on the binding between VEGF-B and FGFR1, and between FGF2 and FGFR1;

FIG. 1D shows the result of an SPR-based competitive binding assay, which reveals the effect on the binding between FGF2 and FGFR1 from VEGF-B or PlGF1;

FIG. 1E shows the result of a dot-blot assay on the binding between VEGF-B and FGFR1 (upper row), and between FGF2 and FGFR1 (middle row); VEGF-B and FGF2 were added in different doses (4.7, 19, 75, 300 and 1200 ng); FGFR-Fc of different doses (1.2, 4.7, 19, 75 and 300 ng) was used as reference (lower row).

FIG. 2A-2D show the results of embodiment 2 of the invention, wherein:

FIG. 2A shows the result of an immunoblot assay on samples from HREC (Human Retinal Endothelial Cells) stimulated with BSA, FGF2, VEGF-B, or FGF2+VEGF-B; the result shows pErk (phosphorylated Erk) and tErk (total Erk) level in the said HREC;

FIG. 2B shows the result of an immunoblot assay on samples from HMVEC (Human Microvascular Endothelial Cells) stimulated with BSA, FGF2, VEGF-B, or FGF2+VEGF-B; the result shows pErk (phosphorylated Erk) and tErk (total Erk) level in the said HMVEC;

FIG. 2C shows the result of an in vivo experiment on retinae from C57B16 mice intravitreally injected with BSA, FGF2, VEGF-B, FGF2+VEGF-B, VEGF-A or VEGF-A+VEGF-B; the result shows pErk (phosphorylated Erk) and tErk (total Erk) level in the said retinae;

FIG. 2D shows the result of an FGFR1 mutant assay; the result shows pErk (phosphorylated Erk) and tErk (total Erk) level in Hela cells transfected with plasmids carrying wild type FGFR1 (FGFR1 WT) or mutated FGFR1 with different mutation sites (lower left).

FIG. 3A-3E show the results of embodiment 3 of the invention, wherein:

FIG. 3A shows the result of a Matrigel angiogenesis in vivo model assay on C57B16 mice injected with Matrigel comprising BSA, FGF2 or FGF2+VEGF-B; microscopic images of H&E (upper left) or CD31 (lower left) immunostaining of fixed Matrigel extracted from the said C57B16 mice are shown; vascular density of the said C57B16 mice are shown (right);

FIG. 3B is a schematic diagram of a gene-knockout strategy, showing the genetic structures of wild type Vegf-b allele (upper part), targeting vector (middle part) and targeted Vegf-b allele (lower part); the result of PCR validation of gene-knockout homozygote (−/−), heterozygote (+/−) and wild type (+/+) was shown (lower right);

FIG. 3C shows the result of a staining of flattened retinae from VEGF-B₁₆₇ deficient mice (right, Vegf-b^{-/-}) and C57B16 mice (left, Vegf-b^{+/+}), along with the percentage of vessel area thereof (right);

FIG. 3D shows the result of an CD31 immunofluorescence assay on endothelial cells extracted from the VEGF-B₁₆₇ deficient mice (right, Vegf-b^{-/-}) and the C57B16 mice (left, Vegf-b^{+/+}), along with the number of CD31 pixels thereof (right);

FIG. 3E shows the result of an aorta ring assay on the aorta ring samples from the VEGF-B₁₆₇ deficient mice (right, Vegf-b^{-/-}) and the C57B16 mice (left, Vegf-b^{+/+}), along with the number of branching per ring thereof (right).

FIG. 4A-4F show the results of embodiment 4 of the invention, wherein:

FIG. 4A shows the result of an immunoblot assay on B16 cells infected by GFP expressing adenoviruses (left, Ad-GFP) or VEGF-B expressing adenoviruses (right, Ad-VEGF-B); the result shows the protein expression of VEGF-B and GFP (stained by Ponceau S);

FIG. 4B shows the result of an subcutaneous tumorigenesis assay on C57B16 mice inoculated with the B16 cells infected by GFP expressing adenoviruses (Ad-GFP) or VEGF-B expressing adenoviruses (Ad-VEGF-B); the result shows the change of tumor volume in the said C57B16 mice over time;

FIG. 4C shows the result of an CD31 immunostaining of tumor samples from the said C57B16 mice inoculated with the B16 cells infected by GFP expressing adenoviruses (Ad-GFP) or VEGF-B expressing adenoviruses (Ad-VEGF-B);

FIG. 4D shows the rate of CD31-positive area in FIG. 4C;

FIG. 4E shows the result of an immunoblot assay on samples from normal liver tissue and liver cancer; the result shows the protein expression level of VEGF-B, FGFR1 and FGFR2 in the said samples;

FIG. 4F shows the result of an immnunoblot assay on samples from normal tissues (endometrial, breast, rectal and bladder tissues) and cancers (endometrial carcinoma, breast, rectal and bladder cancer); the result shows the protein expression level of VEGF-B in the said samples.

FIG. 5A-5G show the results of embodiment 5 of the invention, wherein:

FIG. 5A shows the result of a co-immunoprecipitation assay of interactions between VEGFR1 and FGFR1, and between VEGFR2 and FGFR2 in retina or brain tissue from C57B16 mice;

FIG. 5B shows the result of a co-immunoprecipitation assay on retina samples from C57B16 intravitreally injected with BSA, FGF2 or VEGF-B; the result shows the intensity of the interaction between VEGFR1 and FGFR1 in the said samples;

FIG. 5C shows the result of an in situ proximity ligation assay on HREC (Human Retinal Endothelial Cells) stimulated with BSA, VEGF-B or PlGF; the result shows the signal of FGR1/VEGFR1 complex in the said cells (right), along with the number of the complex per cell in the said cells (upper left);

FIG. 5D shows the result of a fluorescence quantitative real-time PCR which shows the mRNA level of Spry4 in HREC stimulated with BSA (−) or VEGF-B (+);

FIG. 5E shows the result of an immunoblot assay on retinae from C57B16 mice intravitreally injected with BSA or VEGF-B; the result shows Spry4 expression level in the said retinae;

FIG. 5F shows the result of a microarray assay on retinae stimulated with VEGF-B; the result shows the change of the expression level of Spry4 and Spry1;

FIG. 5G shows the result of an in vivo experiment (quantitative real-time PCR) which shows the mRNA level of Spry4 in retinae from C57B16 mice intravitreally injected with BSA or VEGF-B.

FIG. 6A-6D show the results of embodiment 6 of the invention, wherein:

FIG. 6A shows the result of an immunoblot assay on Fgfr1^{flox/flox} mice EC [endothelial cells, having Fgfr1^{flox/flox} knocked out by Cre recombinase expressing adenoviruses (Cre-ad, right) or not (Control-Ad, left)] stimulated with BSA, FGF2, PlGF1, VEGF-B, FGF2+PlGF1 or FGF2+VEGF-B; the result shows pErk (phosphorylated Erk) and tErk (total Erk) level in the said EC;

FIG. 6B shows the result of an immunoblot assay on Fgfr1^{flox/flox} mice EC [endothelial cells, having Flt1^{flox/flox} knocked out by Cre recombinas expressing adenoviruses (Cre-ad, right) or not (Control-Ad, left)] stimulated with BSA, FGF2, PlGF1, VEGF-B, FGF2+PlGF1 or FGF2+VEGF-B; the result shows pErk (phosphorylated Erk) and tErk (total Erk) level in the said EC;

FIG. 6C shows the result of an immunoblot assay on retinae from Spry4^{-/-} or Spry4^{+/+0} mice intravitreally injected with BSA, FGF2, VEGF-B or FGF2+VEGF-B; the result shows pErk (phosphorylated Erk) and tErk (total Erk) level in the said retinae;

FIG. 6D is a schematic diagram showing that the VEGF-B/FGFR1 signaling pathway promotes the up-regulation of Spry4 expression, and antagonizes the FGF2-promoted neoangiogenesis;

FIG. 7A-7F show the results of embodiment 7 of the invention, wherein:

FIG. 7A shows the result of an SPR (Surface Plasmon Resonance) assay on the binding between VEGF-B and FGFR2, and between FGF2 and FGFR2;

FIG. 7B shows the result of a pull-down experiment on VEGF-B (0, 0.05, 0.1, 0.3, 0.6, 0.9 and 1.5 μg) and FGF2 (0, 0.01, 0.05, 0.1, 0.3, 0.5 and 0.8 μg) with FGFR2 (FGFR2-Fc);

FIG. 7C shows the result of an alkaline phosphatase assay of FGFR2 on COS-7 cells transfected with FGFR2-AP (alkaline phosphatase) expressing plasmid;

FIG. 7D shows the result of a dot-blot assay which detects the binding between VEGF-B and FGFR2, and between FGF2 and FGFR2;

FIG. 7E shows the result of an in situ proximity assay on HUVSMC stimulated by BSA, VEGF-B or PlGF; the result shows the signal of VEGF-B/FGFR2 complex, along with the counts of the complex per cell in the said HUVSMC (lower right);

FIG. 7F shows the result of a dynamic assay on the binding between VEGF-B and FGFR2; the result shows the change of the optical density (OD) of FGFR2-binding VEGF-B as the concentration of VEGF-B increases.

FIG. 8A-8E show the results of embodiment 8 of the invention, wherein:

FIG. 8A shows the result of antibody chip assay detecting the phosphorylation of FGFR2 or FGFR3;

FIG. 8B shows the result of a co-immunoprecipitation assay on HUVSMC stimulated with BSA, FGF2, PlGF1 or VEGF-B; the result shows the level of FGFR2 (lower row) and FGFR2 with phosphorylated tyrosine residues (pTyr, upper row) in the said HUVSMC;

FIG. 8C shows the result of a co-immunoprecipitation assay on HREC stimulated with FGF2 or VEGF-B for different time lengths (0, 10, 30, 60 and 120 mins; the result shows the level of FGFR2 (lower row) and FGFR2 with phosphorylated tyrosine residues (pTyr, upper row) in the said HREC;

FIG. 8D shows the result of a co-immunoprecipitation assay on HMVEC and PAE-FGFR2c stimulated with BSA, FGF2 or VEGF-B; the result shows the level of FGFR2 (lower row) and FGFR2 with phosphorylated tyrosine residues (pTyr, upper row) in the said HMVEC and PAE-FGFR2c;

FIG. 8E shows the result of a co-immunoprecipitation assay on PC3 and OVCAR4 stimulated with BSA, FGF2 or VEGF-B; the result shows the level of FGFR2 (lower row) and FGFR2 with phosphorylated tyrosine residues (pTyr, upper row) in the said PC3 and OVCAR4.

FIG. 9A-9C show the results of embodiment 9 of the invention, wherein:

FIG. 9A shows the result of a co-immunoprecipitation assay of interactions between FGFR2 and VEGFR1, and between FGFR2 and VEGFR2 in brain tissue and retina from C57B16 mice;

FIG. 9B shows the result of a co-immunoprecipitation assay of interaction between FGF2 and VEGFR1 in retinae from C57B16 mice intravitreally injected with BSA, FGF2 or VEGF-B;

FIG. 9C shows the result of an in situ proximity ligation assay on SMC (Mouse Primary Smooth Muscle Cells) stimulated with BSA, VEGF-B or PlGF; the result shows the signal of FGFR2/VEGFR1 complex, along with the counts of the complex per cell in the said SMC.

FIG. 10A-10G show the results of embodiment 10 of the invention, wherein:

FIG. 10A shows the result of a real-time quantitative PCR detecting the mRNA level of Spry4 in HUVSMC stimulated with VEGF-B₁₆₇ for different time lengths (0, 10 mins, 30 mins, 1 hr, 2 hrs and 6 hrs);

FIG. 10B shows the result of an immunoblot assay on HUVSMC stimulated with BSA or VEGF-B; the result shows the protein expression level of Spry4 in the said HUVSMC;

FIG. 10C shows the result of an immunoblot assay on Flt1^{flox/flox} SMC [having Flt1^{flox/flox} knocked out by Ad-Cre (+) or not (−)] stimulated with BSA (−) or VEGF-B (+); the result shows the protein expression level of Spry4 in the said SMC;

FIG. 10D shows the result of an immunoblot assay on Fgfr2^{flox/flox} SMC [having Fgfr2^{flox/flox} knocked out by Ad-Cre (+) or not (−)] stimulated with BSA (−) or VEGF-B (+); the result shows the protein expression level of Spry4 in the said SMC;

FIG. 10E shows the result of an immunoblot assay detecting the protein expression level of Spry4 in HUVSMC stimulated with BSA, FGF2 or VEGF-B;

FIG. 10F shows the result of an immunoblot assay detecting the protein expression level of Spry4 in endothelial cells EAhy926 stimulated with BSA or VEGF-B for different time lengths (24 hrs and 48 hrs);

FIG. 10G shows the result of an immunoblot assay detecting the protein expression level of Spry4 in OVCAR4 stimulated with BSA, or with VEGF-B for different time lengths (6, 12, 20, 30 and 40 hrs).

FIG. 11A-11G show the results of embodiment 11 of the invention, wherein:

FIG. 11A shows the result of an immunoblot assay detecting pErk (phosphorylated Erk) and tErk (total Erk) level in HUVSMC stimulated with BSA, FGF2, FGF2+PlGF1 or FGF2+VEGF-B;

FIG. 11B shows the result of an immunoblot assay detecting pErk (phosphorylated Erk) and tErk (total Erk) level in SMC stimulated with BSA, FGF2, PlGF1, VEGF-B, FGF2+PlGF1 or FGF2+VEGF-B;

FIG. 11C shows the result of an immunoblot assay detecting pErk (phosphorylated Erk) and tErk (total Erk) level in Flt1^{flox/flox} SMC [having Flt1^{flox/flox} knocked out by Ad-Cre (+) or not (−)] stimulated with BSA, FGF2, PlGF1, VEGF-B, FGF2+PlGF1 or FGF2+VEGF-B;

FIG. 11D shows the result of an immunoblot assay detecting pErk (phosphorylated Erk) and tErk (total Erk) level in Flt1-tk^{+/+} (wild type) SMC and Flt1-tk^{-/-} SMC stimulated with BSA, FGF2, PlGF1, VEGF-B, FGF2+PlGF1 or FGF2+VEGF-B;

FIG. 11E shows the result of an immunoblot assay detecting pErk (phosphorylated Erk) and tErk (total Erk) level in Fgfr2^{flox/flox} SMC [having Fgfr2^{flox/flox} knocked out by Ad-Cre (+) or not (−)] stimulated with BSA, FGF2, PlGF1, VEGF-B, FGF2+PlGF1 or FGF2+VEGF-B;

FIG. 11F shows the result of an immunoblot assay detecting pErk (phosphorylated Erk) and tErk (total Erk) level in Spry4^{+/+} (wild type) SMC and Spry4^{-/-} SMC stimulated with BSA, FGF2, PlGF1, VEGF-B, FGF2+PlGF1 or FGF2+VEGF-B;

FIG. 11G shows the result of an immunoblot assay detecting pErk (phosphorylated Erk) and tErk (total Erk) level in PAE-FGFR2 stimulated with BSA, FGF2, PlGF1, VEGF-B, FGF2+PlGF1 or FGF2+VEGF-B.

FIG. 12A-12E show the results of embodiment 12 of the invention, wherein:

FIG. 12A shows the result of a cell proliferation assay on HUVSMC stimulated with BSA, FGF2, FGF2+VEGF-B, FGF2+PlGF1, VEGF-B or PlGF1; the result shows the proliferation indexes of the said HUVSMC;

FIG. 12B shows the images (left) and counts (right) of migrating cells of a cell migration assay on HUVSMC stimulated with BSA, FGF2, FGF2+VEGF-B or VEGF-B;

FIG. 12C shows the images (left) and rate of SMA-positive field (right) of an SMA+DAPI staining of sectioned retinae from C57B16 mice (WT) and VEGF-B deficient mice (Vegf-b^{-/-}); the marked retinal layers include INL (inner nuclear layer) and ONL (outer nuclear layer);

FIG. 12D shows the images (left) and rate of NG2-positive field (right) of an NG2+IB4+DAPI staining of sectioned retinae from C57B16 mice (WT) and VEGF-B deficient mice (Vegf-b^{-/-}); the marked retinal layers include RGCL (retinal ganglion cell layer), INL (inner nuclear layer) and ONL (outer nuclear layer);

FIG. 12E is a schematic diagram showing that the VEGF-B/FGFR2 signaling pathway promotes the up-regulation of Spry4 expression, and antagonizes the FGF2-promoted phosphorylation of Erk.

It is to be noted, that all the “VEGF-B” showed in the drawings refer to VEGF-B₁₆₇.

### DETAILED DESCRIPTION OF ILLUSTRATED EMBODIMENTS

In order to better illustrate the purpose, technical scheme and advantages of the invention, the invention will be further illustrated in conjunction with the drawings and embodiments.

As used herein, unless specified otherwise, the terms “HREC”, “HUVSMC”, “HMVEC”, “PAE” are described as follows:

“HREC” refers to “Human Retinal Endothelial Cells”;

“HUVSMC” refers to “Human Umbilical Vein Smooth Muscle Cells”;

“HMVEC” refers to “Human Microvascular Endothelial Cells”;

“PAE” refers to “Porcine Aortic Endothelial Cells”;

“OVCAR4” refers to “Human Ovarian Cancer Cells”;

“SMC”, “mouse primary SMC” and similar terms refer to “Mouse Primary Smooth Muscle Cells”.

As used herein, unless specified otherwise, BSA (Bovine Serum Albumin) was used as blank control in all assays or experiments (i.e., intravitreal injection, cell stimulation). β-actin and GAPDH (expression level) were used as internal reference in all assays or experiments (i.e., immunoblot assay).

It is to be noted that, as many of the following embodiments adopt same experimental assays (i.e., immunoblot assay, Real-time quantitative PCR, in situ proximity ligation assay, co-immunoprecipitation assay, microarray assay), the detailed steps or operational procedures of these assays are only briefly described in the latter embodiments.

### Embodiment 1: VEGF-B₁₆₇ Binds to and Activates FGFR1

Experimental Materials:

HREC (Human Retinal Endothelial Cells) and HUVSMC (Human Umbilical Vein Smooth Muscle Cells).

Experimental Methods:

Immunoblot assay (Western-blot): HREC and HUVSMC were conventionally cultured. Respectively, FGF2 (50 ng/ml) or VEGF-B (100 ng/ml) was added for a 15-minute or 30-minute stimulation, proteins were then extracted. SDS-PAGE was performed to analyze levels of phosphorylated FGFR1 (pFGFR1) and total FGFR1 (tFGFR1).

Surface plasmon resonance (SPR) assay: FGFR1-Fc was fixed on a sensor, FGF2 or VEGF-B₁₆₇ was then added to analyze their binding to FGFR1.

SPR-based competitive binding assay: FGFR1-Fc was fixed on a sensor VEGF-B₁₆₇ or PlGF1 of different concentrations (respectively 10 ng/ml, 50 ng/ml, 100 ng/ml, 200 ng/ml, 500 ng/ml, 1000 ng/ml) was added, FGF2 was then added to analyze competitive inhibition to bindings between FGF2 and FGFR1 of VEGF-B₁₆₇ or PlGF1.

Dot-blot assay: Human VEGF-B₁₆₇ or FGF2 protein (as positive control) of different doses (4.7, 19, 75, 300 and 1200 ng) were respectively dotted on a upper row and a middle row of a film, FGFR1c-Fc protein of different doses (1.2, 4.7, 19, 75 and 300 ng) were dotted on a lower row. 1 μg/ml FGFR1c-Fc was added to the film blocked by BSA for incubation, the film was further incubated by peroxidase-labeled human IgG Fcγ to color.

Experimental Results:

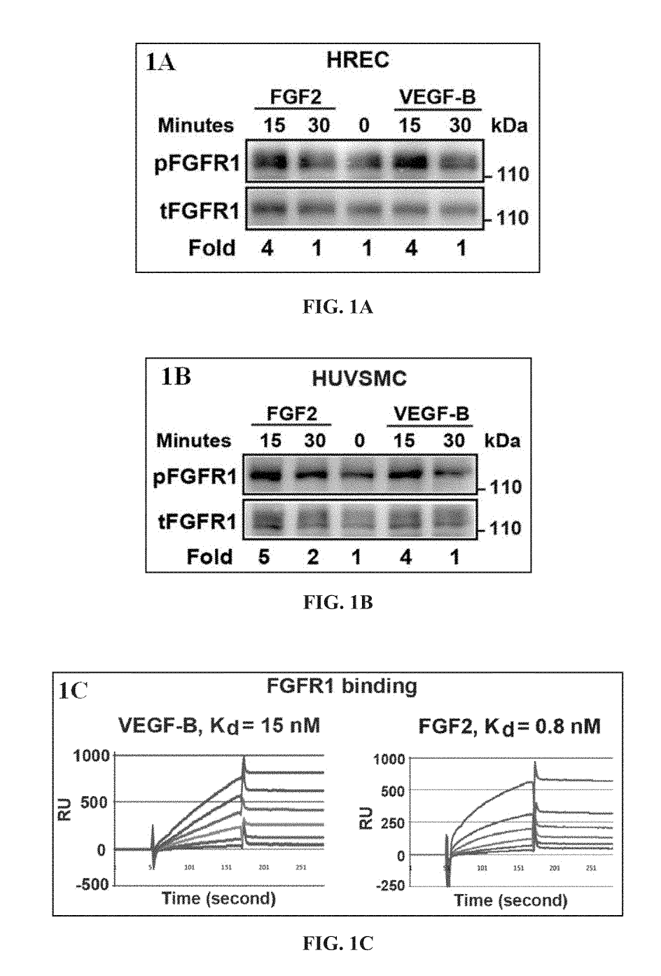

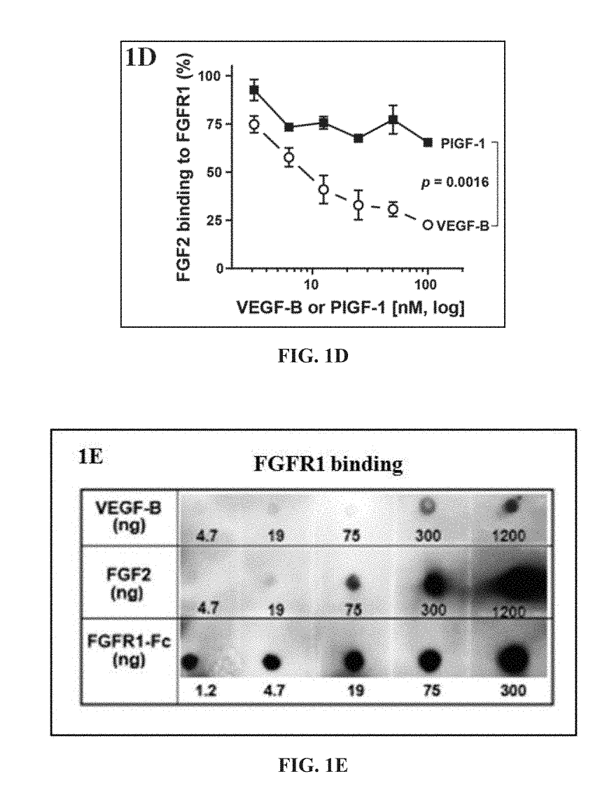

As illustrated in FIG. 1A-1E, wherein FIG. 1A shows the result of the immunoblot assay, it is shown that VEGF-B₁₆₇ activated FGFR1 in HREC; FGF2 was used as a positive control.

FIG. 1B shows that VEGF-B₁₆₇ induced the phosphorylation of FGFR1.

FIG. 1C shows the result of the SPR assay, that VEGF-B₁₆₇ binds to FGFR1 with a Kd value of 15 nM.

FIG. 1D shows the result of the competitive binding assay, that VEGF-B₁₆₇ competed with FGF2 for binding to FGFR1, while PlGF1 could not.

FIG. 1E shows the result of the dot-blot assay. The result shows that VEGF-B₁₆₇ binds to FGFR1.

### Embodiment 2: VEGF-B₁₆₇ Inhibits FGF2 from Activating Erk

Experimental Materials:

HREC (Human Retinal Endothelial Cells), HMVEC (Human Microvascular Endothelial Cells), Hela (human cervical cancer cells) and 8-week-old C57B16 mice.

Experimental Methods:

Immunoblot assay (Western-blot): HREC and HMVEC were conventionally cultured. BSA, FGF2 (50 ng/ml), VEGF-B₁₆₇ (100 ng/ml) or FGF2 (50 ng/ml)+VEGF-B₁₆₇ (100 ng/ml) was added for a 15-minute stimulation, proteins were then extracted. SDS-PAGE was performed to analyze levels of phosphorylated Erk (pErk) and total Erk (tErk).

In vivo experiment: C576B16 mice were intravitreally injected with BSA, FGF2, VEGF-B, FGF2+VEGF-B, VEGF-A or VEGF-A+VEGF-B, proteins were extracted from the retinae 30 minutes after. Western-blot was performed to analyze levels of phosphorylated Erk.

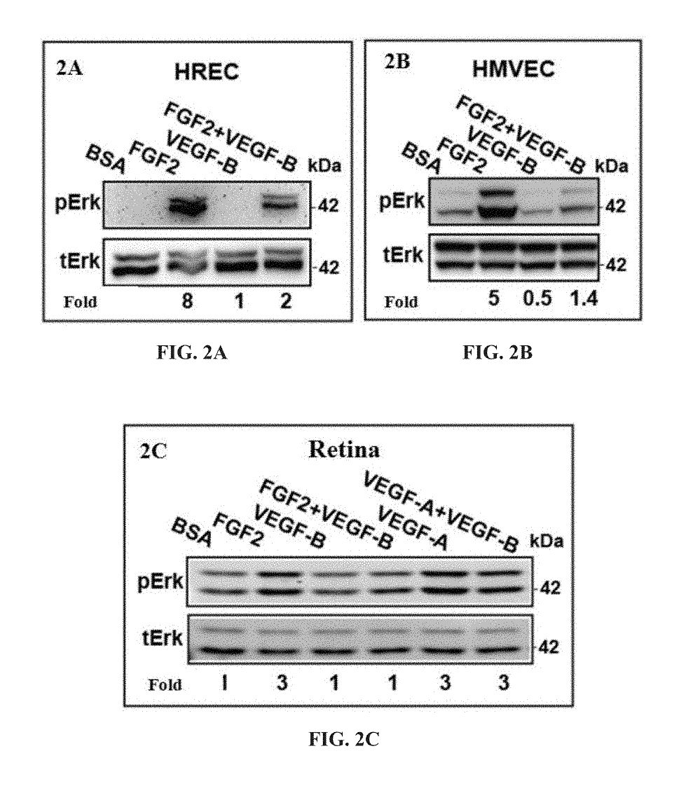

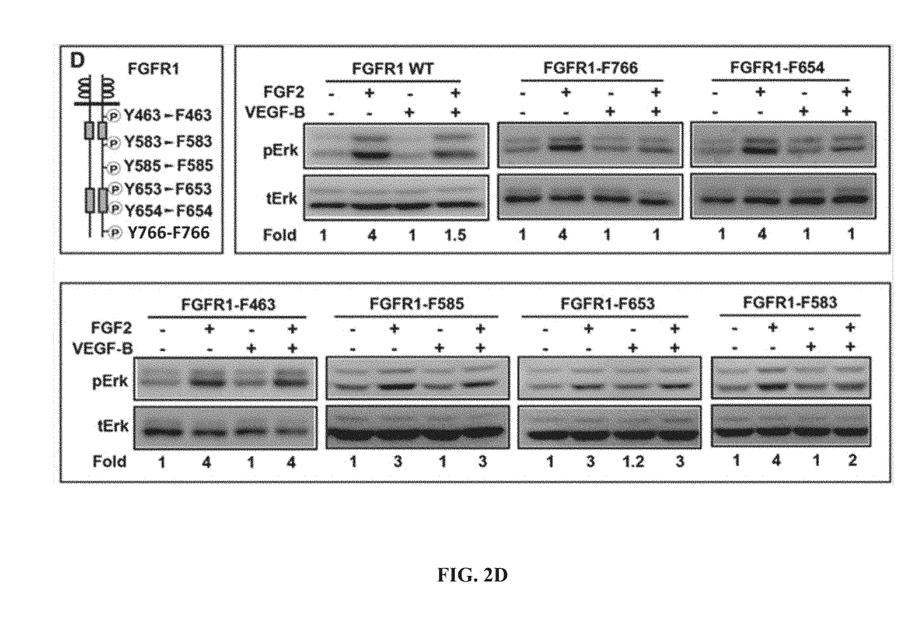

FGFR1 mutant assay: Hela cells were transfected with plasmids carrying wild type FGFR1 (FGFR1 WT) or mutated FGFR1 with different mutation sites (as shown in FIG. 2D, lower left), FGF2 and VEGF-B₁₆₇ were then added individually or in combination for stimulation, levels of phosphorylated Erk were analyzed to find a target site by which FGFR1 interacts with VEGF-B₁₆₇.

Experimental Results:

As illustrated in FIG. 2A and FIG. 2B, FGF2 induced the phosphorylation of Erk in HREC (FIG. 2A) and HMVEC (FIG. 2B). Such induction was weakened after the addition of VEGF-B₁₆₇.

In the in vivo experiment (FIG. 2C), the retinae of the C57B16 mice were used for immunoblot assay. It is shown that FGF2 was inhibited from inducing the phosphorylation of Erk by injecting VEGF-B₁₆₇ into the vitreous bodies of the mice, while the induction of the phosphorylation of Erk by VEGF-A was not affected by the VEGF-B₁₆₇ injection.

As illustrated in FIG. 2D, intracytoplasmic tyrosine residues of FGFR1 were phosphorylated when FGFR1 was activated; VEGF-B₁₆₇ inhibited FGF2 from inducing the phosphorylation of Erk when wild type (WT) FGFR1 or FGFR1 mutants (F766 and F654) were individually transfected.

As illustrated in FIG. 2D, the inhibition of VEGF-B₁₆₇ on the phosphorylation of Erk induced by FGF2 disappeared when other FGFR1 mutants were individually transfected (F463, F585, F653 and F583).

### Embodiment 3: VEGF-B₁₆₇ Inhibits FGF2-Induced Neoangiogenesis

Experimental Materials:

8-week-old C57B16 mice, Matrigel (356230, BD Bioscience) and VEGF-B gene deficient mice.

Experimental Methods:

Matrigel angiogenesis in vivo model assay: 0.5 ml Matrigel comprising heparin (10 μg/ml) and BSA (300 ng/ml, Sigma), FGF2 (150 ng/ml, PeproTech), VEGF-B₁₆₇ (300 ng/ml, PeproTech) or FGF2 (150 ng/ml)+VEGF-B₁₆₇ (300 ng/ml) was injected subcutaneously into abdomens of the C57B16 mice. The C57B16 mice were sacrificed 7 days after. The Matrigel was extracted, then fixed with 4% PFA and sectioned, and then immunostained by H&E or CD31.

VEGF-B gene deficient mice assay: the VEGF-B gene deficient mice obtained by gene-knockout technique were validated by PCR. Retinae of the VEGF-B gene deficient mice were extracted, flattened and immunostained by H&E or CD31. Brain tissue of the VEGF-B gene deficient mice was extracted, sectioned and immunostained by CD31.

Mouse aortic ring assay: aortas of the C57B16 and VEGF-B₁₆₇ gene deficient mice were separated with exterior adipose and connective tissues carefully removed, and were cut into 1.0 mm long each. The aortic rings were then put into serum-free medium in an incubator with 5% CO₂ at 37° C. for overnight starvation. On the second day, the aortic rings were seeded in Matrigel, and FGF2 (20 ng/ml) was added. The medium was changed every two days. Images were collected 14 days after for vascular quantification.

Experimental Results:

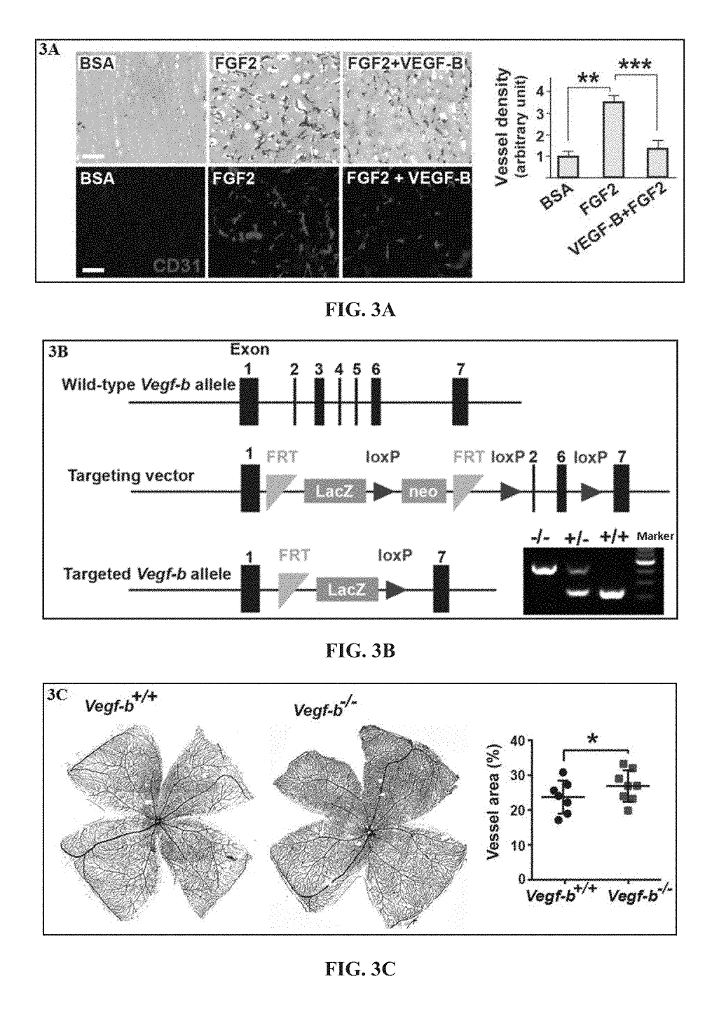

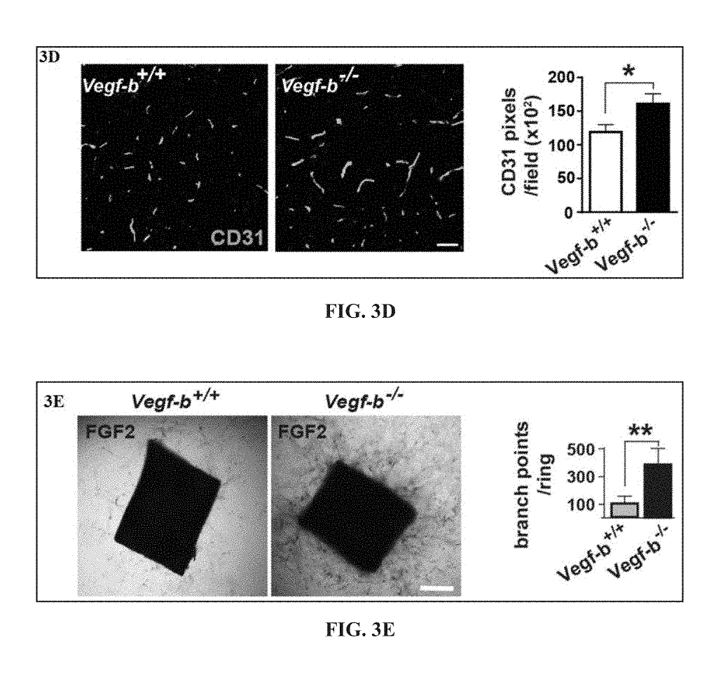

As illustrated in FIG. 3A-3E, the matrigel angiogenesis in vivo model assay shows that VEGF-B₁₆₇ inhibited the FGF2-induced neoangiogenesis.

The schematic of gene-knockout strategy (FIG. 3B) shows that a LacZ cassette replaces a genomic segment covering from exons 2 to 6 of Vegf-b gene. Mice genotypes of gene-knockout homozygote (−/−), heterozygote (+/−) and wild type (+/+) were validated by PCR.

The stained flattened retinae (FIG. 3C) show an increased vascular density of the retinae in the VEGF-B₁₆₇ gene deficient mice.

Immunofluorescence assay (FIG. 3D) detected marker protein CD31 of endothelial cells, the result shows an increased vascular density of the brains of the VEGF-B₁₆₇ gene deficient mice.

The result of the aorta ring assay (FIG. 3E) shows a significant increase in the FGF2-induced neoangiogenesis in the VEGF-B₁₆₇ gene deficient aortic rings, indicating that VEGF-B₁₆₇ inhibits FGF2-induced neoangiogenesis, and that the deficiency of VEGF-B₁₆₇ caused increased neoangiogenesis.

### Embodiment 4: VEGF-B₁₆₇ Inhibits Tumor Growth and Neoangiogenesis &VEGF-B₁₆₇ Expression Decreases in Tumor Tissues

Experimental Materials:

VEGF-B₁₆₇ adenovirus expression vectors, B16 cells (melanoma cells), tumor tissue samples of liver cancer, endometrial cancer, breast cancer, bladder cancer and rectal cancer, and 8-week-old C57B16 mice.

EXPERIMENTAL methods:

Subcutaneous tumorigenesis assay: the VEGF-B₁₆₇ adenovirus expression vectors (Ad-VEGF-B) (GFP expression vectors used in a control group, Ad-GFP) was co-incubated with the B16 cells for 1 hour. Each of the C57B16 mice was subcutaneously inoculated with 10⁶ cells. Tumor size was measured 13-17 days after the inoculation, and the C57B16 mice were sacrificed on the 17^{th} day. The tumor tissue was taken, sectioned and stained by CD31.

Immunoblot assay (western-blot): the tumor tissue samples of liver cancer, endometrial cancer, breast cancer, bladder cancer and rectal cancer were homogenated, and the supernatant was collected for protein quantification, immunoblot assay of VEGF-B₁₆₇ expression was performed. Another immunoblot assay of VEGF-B₁₆₇ expression was performed on the B16 cells transfected with Ad-GFP or Ad-VEGF-B for validation.

Experimental Results:

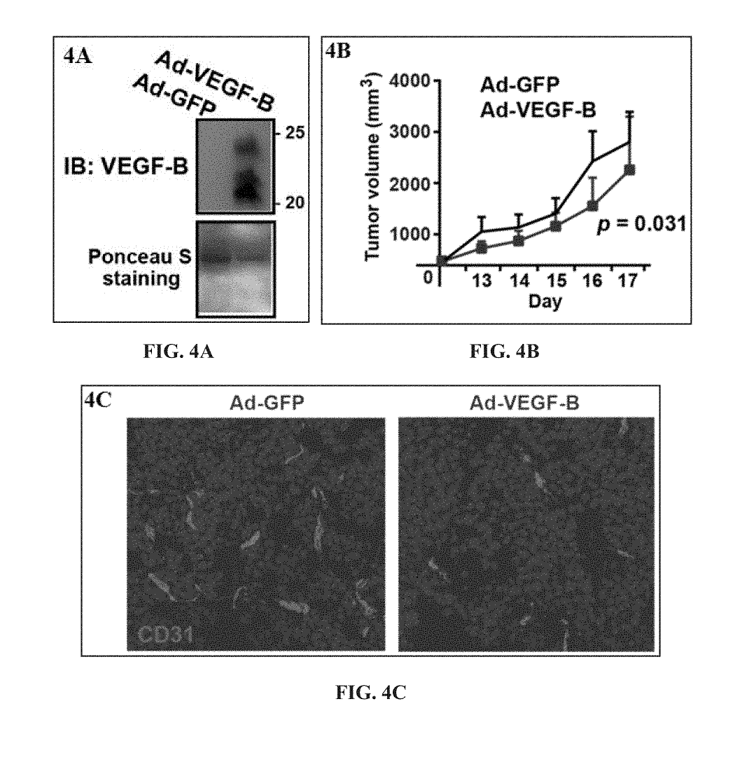

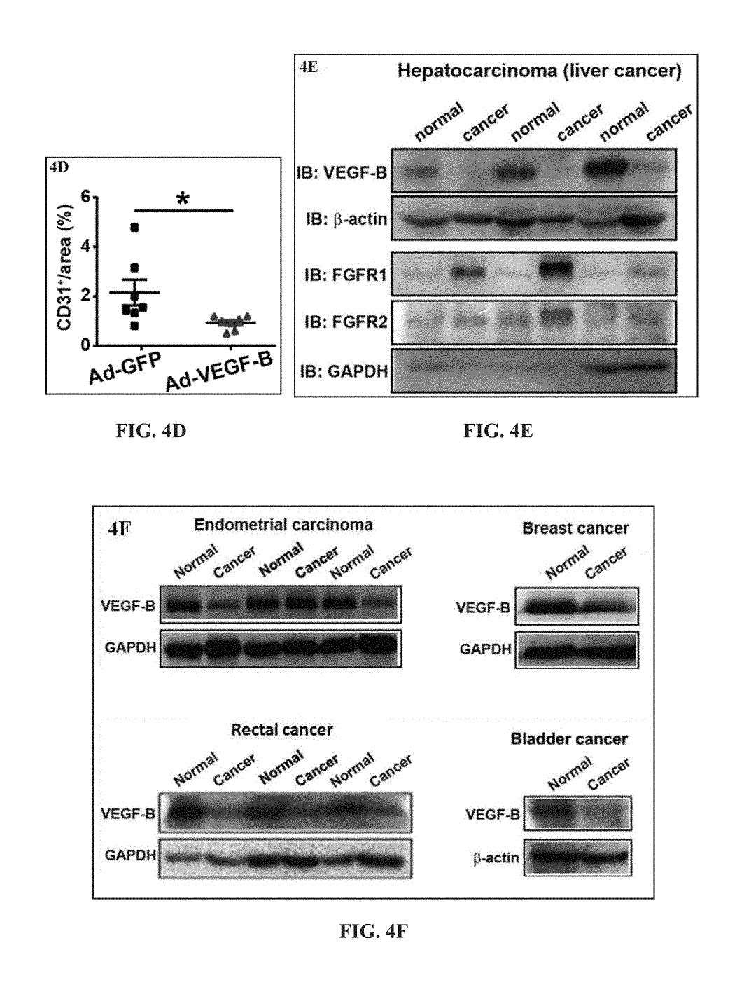

As illustrated in FIG. 4A-4F, VEGF-B₁₆₇ was overexpressed by the adenovirus expression vectors in the B16 cells, and the result of the immunoblot assay (FIG. 4A) indicates the overexpression of VEGF-B₁₆₇ in the B16 cells.

The result of the subcutaneous tumorigenesis assay (FIG. 4B) shows that the overexpressed VEGF-B₁₆₇ inhibited the growth of B16 tumor.

The immunofluorescence staining detected vascular marker protein CD31, and the result (FIG. 4C) shows that VEGF-B₁₆₇ significantly decreased the number of blood vessels in the tumor. FIG. 4D is a statistical scatter diagram of vascular density (percentage of CD31-positive area) of the result in FIG. 4C.

The immunoblot assay (FIG. 4E) detected the protein expression levels of VEGF-B₁₆₇, FGFR1 and FGFR2 in the clinical samples of liver cancer, and the result shows that the protein expression levels of VEGF-B₁₆₇ in the liver tumor samples were significantly lower than those in the normal liver samples, and that the expression level of VEGF-B₁₆₇ was negatively correlated with those of FGFR1 and FGFR2. The expression levels of GAPDH and β-actin were used as internal reference.

The immunoblot assay (FIG. 4F) also indicates that the protein expression levels of VEGF-B₁₆₇ in tissues of endometrial cancer, breast cancer, bladder cancer and rectal cancer, are lower than those in normal tissues. The expression levels of GAPDH and β-actin were used as internal reference.

### Embodiment 5: VEGF-B₁₆₇ Induces VEGFR1 and FGFR1 to Form a Complex & VEGF-B Upregulates Spry4 Expression

Experimental Materials:

8-week-old C57B16 mice, HREC and Duolink II PLA kit (Sigma, DUO92007).

Experimental Methods:

Co-immunoprecipitation assay: brain tissues and retinae of the C57B16 mice were separated, and were homogenated after added with RIPA buffer comprising protease and phosphatase inhibitor. Supernatant was obtained by centrifuging. After protein quantification, the supernatant was incubated with anti-FGFR1 antibody at 4° C. overnight. Magnetic beads combined with A/G protein were added to capture the antibody complex. 10% PAGE electrophoresis and transfer to a PVDF film were performed successively for the complex, then the complex was incubated with the primary antibody of VEGFR1, VEGFR2 or FGFR1, and was further incubated with HRP-labeled secondary antibody, ECL luminescence reagent was added to display color in the end.

In order to specify the function of VEGF-B₁₆₇, the C57B16 mice were intravitreally injected with VEGF-B (500 ng/eye), FGF2 (100 ng/eye) or BSA (500 ng/eye), the retinae were separated) hour later for another same co-immunoprecipitation assay.

Immunoblot assay: the C57B16 mice were intravitreally injected with VEGF-B (500 ng/eye) or BSA (500 ng/eye), the retinae were separated 2 or 24 hours later for immunoblot assay.

In situ proximity ligation assay: the operation was performed according to the instruction of Duolink II PLA kit (Sigma, DUO92007). HREC were stimulated with BSA, VEGF-B₁₆₇ or PlGF, fixed with 4% PFA, and were added with Anti-FGFR1 antibody and anti-VEGF-B₁₆₇ antibody. Further incubation with secondary antibodies of Duolink II anti-mouse plus and Duolink II anti-rabbit minus was performed to display color. Images were taken.

Fluorescence quantitative real-time PCR: HREC was stimulated with BSA or VEGF-B₁₆₇. The HREC were lysed by TRIZOL, and the total RNA was extracted. 3 μg total RNA was reversely transcribed, the obtained cDNA was used as a template for PCR to detect Spry4 expression.

Gene microarray assay: A gene microarray assay of Spry1 and Spry4 expression was performed on the said retinae used for the immunoblot assay.

Real-time quantitative PCR: A real-time qPCR of Spry4 expression was performed on the said retinae used for the immunoblot assay.

Experimental Result:

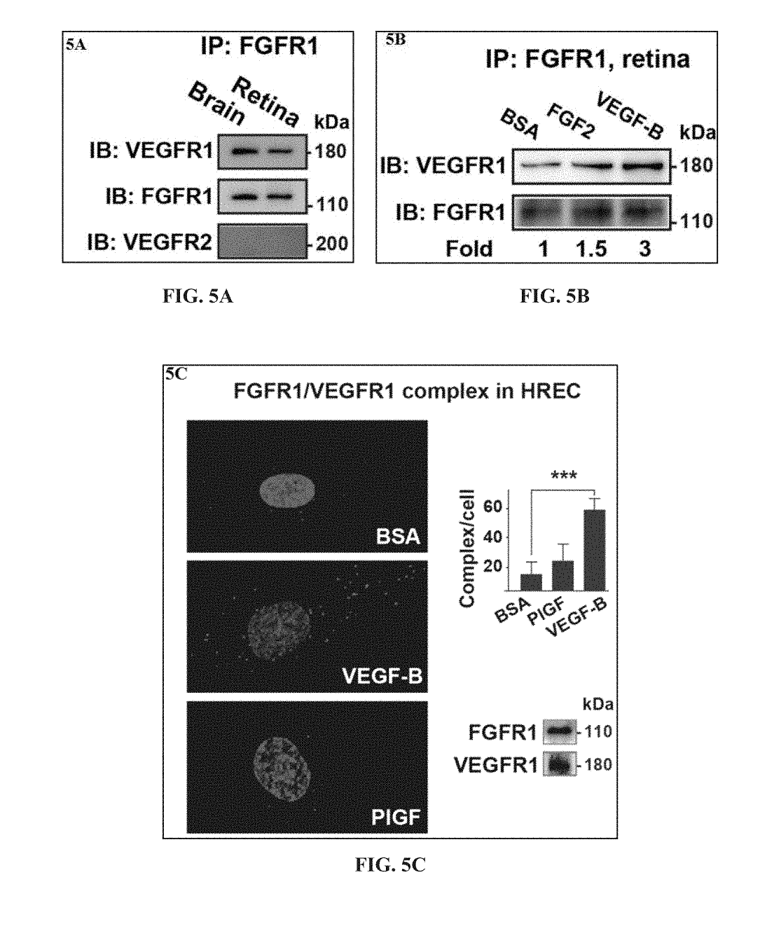

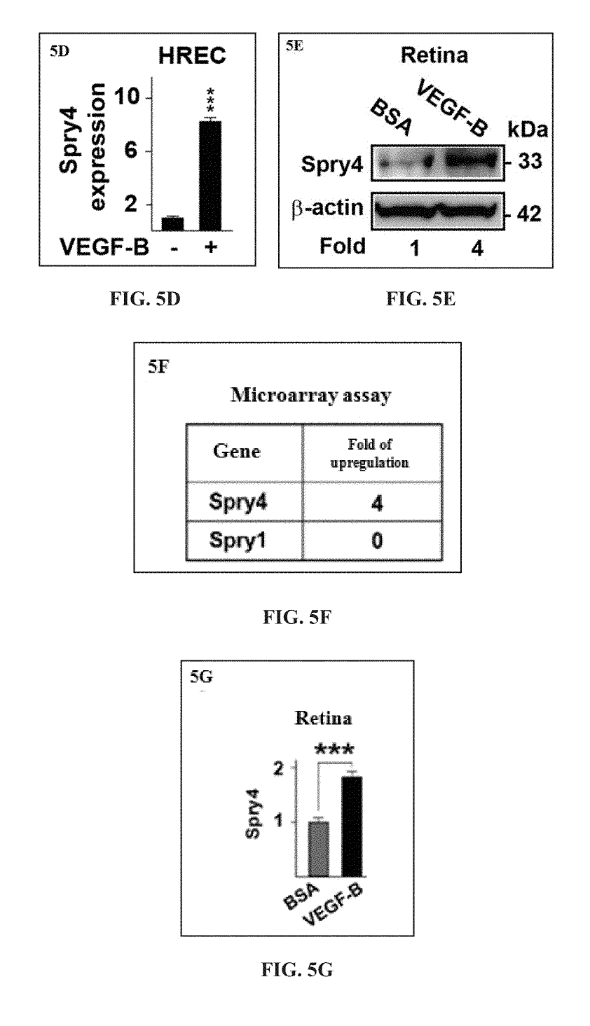

As illustrated in FIG. 5A-5G, the result of the co-immunoprecipitation assay (FIG. 5A) shows that in the retina and brain tissue of the C57B16 mice, FGFR1 bound to VEGFR1 to form a complex, while FGFR1 had no interaction with VEGFR2.

The result of the co-immunoprecipitation assay (FIG. 5B) also indicates that interaction between FGFR1 and VEGFR1 was enhanced by the injection of VEGF-B₁₆₇ into the vitreous bodies of the mice.

The result of the in situ proximity ligation assay (FIG. 5C) shows that VEGF-B₁₆₇ induced the formation of a FGFR1/VEGFR1 complex in HREC, and that PlGF1 caused no significant inducement on the formation of such complex. This indicates that VEGF-B₁₆₇ has specificity for such inducement.

The result of the fluorescence quantitative real-time PCR (FIG. 5D) shows that VEGF-B₁₆₇ up-regulated Spry4 expression in HREC.

The result of the immunoblot assay (FIG. 5E) confirms that Spry4 expression in the retinae was up-regulated by the injection of VEGF-B₁₆₇ into the vitreous bodies of the mice.

The result of gene microarray (FIG. 5F) shows that VEGF-B up-regulated Spry4 expression (4 folds), while Spry1 expression was unaffected.

The result of the Real-time qPCR also shows an increase in Spry4 expression after the injection of VEGF-B₁₆₇ (FIG. 5G).

### Embodiment 6: VEGF-B₁₆₇ Inhibits FGF2 from Promoting Neoangiogenesis and Growth Via FGFR1, Flt1 and Spry4

Experimental Materials:

Fgfr1^{flox/flox} mice, Flt1^{flox/flox} mice, Spry4-knockout mice (Spry4^{-/-}), wild type mice (Spry4^{+/+}) of a litter and Cre recombinase expressing adenoviruses (Cre-Ad).

Experimental Methods:

Extraction of mouse primary endothelial cells (EC): Hearts from 4 mice (Fgfr1^{flox/flox}, Flt1^{flox/flox}, Spry4^{-/-}, Spry4^{+/+}) were shredded and added with a solution of collagenase I for a 45-minute digestion at 37° C., the cells were blown into single-cell suspension. CD31-combined magnetic beads were added for a 15-minute incubation at room temperature. After washed, the cells binding to the beads were transferred to a gelatin-coated culture dish and cultured in ECM medium containing ECGS.

Knockout of flox/flox gene by Cre recombinase expressing adenoviruses (Cre-Ad): the primary endothelial cells (EC) from the Fgfr1^{flox/flox} mice and Flt1^{flox/flox} mice were infected by the Cre recombinase expressing adenoviruses (Cre-Ad) for 48 hours to knockout the flox/flox gene. Control-Ad was used as control.

Immunoblot assay: the Fgfr1^{flox/flox} and Flt1^{flox/flox} mouse primary EC (having their flox/flox gene knocked out by Cre-Ad or not) were stimulated with BSA, FGF2, PlGF1, VEGF-B, FGF2+PlGF1 or FGF2+VEGF-B, and used for an immunoblot assay to detect pErk and tErk level.

The Spry4^{-/-} and Spry4^{+/+} mouse primary EC were stimulated with BSA, FGF2, VEGF-B or FGF2+VEGF-B, and used for an immunoblot assay to detect pErk and tErk level.

Experimental Results:

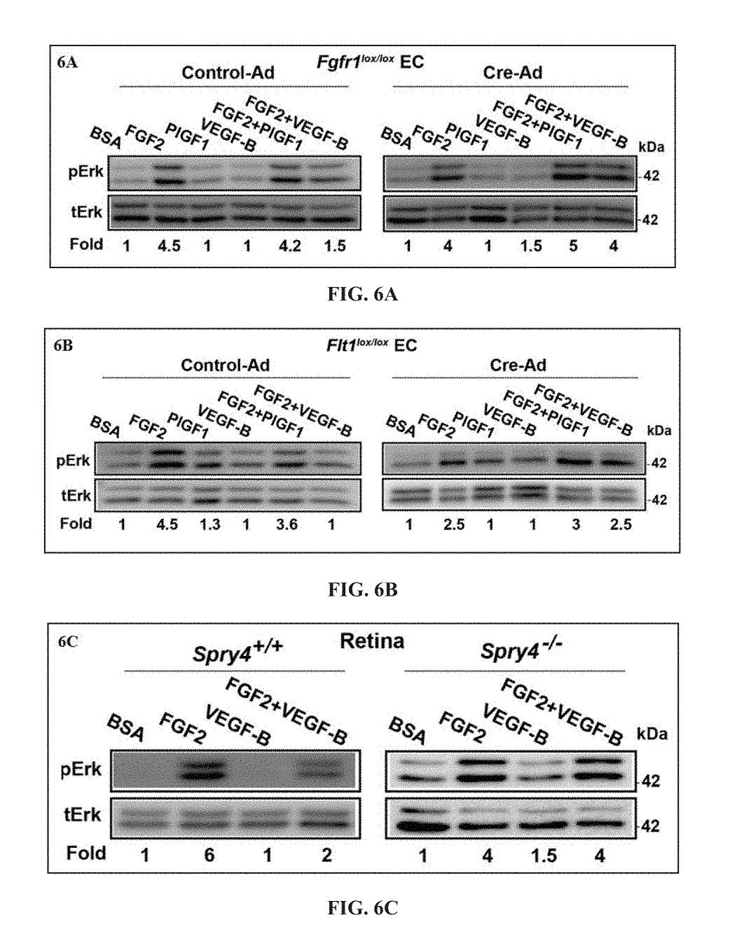

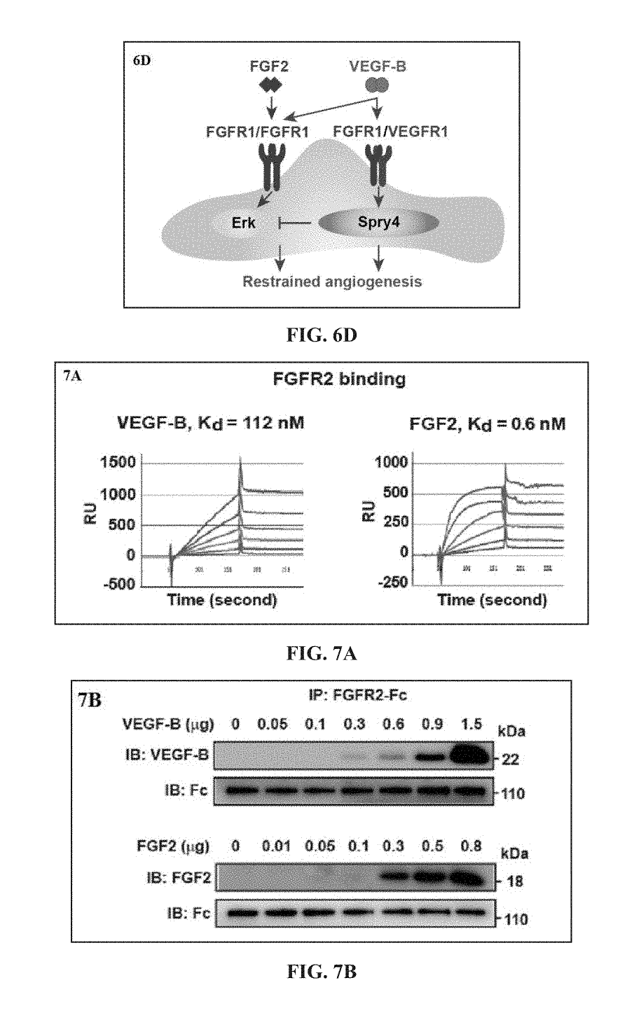

As illustrated in FIG. 6A, it is shown that in the primarily cultured wild type (with normal FGFR1 expression) mouse vascular endothelial cells, VEGF-B₁₆₇ inhibited FGF2 from activating Erk, while PlGF1 had no such inhibition, indicating that VEGF-B₁₆₇ specifically inhibits FGF2 from activating Erk. Such inhibition from VEFG-B₁₆₇ was blocked when FGFR1 gene was knocked out from the endothelial cells by the adenovirus vectors expressed Cre recombinase.

As illustrated in FIG. 6B, it is shown that in the primarily cultured wild type (with normal Flt1 expression) mouse vascular endothelial cells, VEGF-B₁₆₇ inhibited FGF2 from activating Erk, while PlGF1 had no such inhibition, indicating that VEGF-B₁₆₇ specifically inhibits FGF2 from activating Erk. Such inhibition from VEFG-B₁₆₇ was blocked when Flt1 gene was knocked out from the endothelial cells by the adenovirus vectors expressed Cre recombinase.

As illustrated in FIG. 6C, FGF2 was inhibited from activating the retinal Erk by the injection of VEGF-B₁₆₇ into the vitreous bodies of the wild type (Spry4+/+) mice, while VEGF-B₁₆₇ had no effect of such inhibition in a same experimental model established by Spry4 gene deficient mice (Spry4−/−).

In summary, the VEGF-B/FGFR1 signaling pathway promotes the up-regulation of Spry4 expression, and antagonizes the FGF2-promoted neoangiogenesis (FIG. 6D). Therefore, VEGF-B₁₆₇ has critical inhibition on the FGF2-promoted neoangiogenesis.

### Embodiment 7: VEGF-B₁₆₇ Binds to FGFR2

Experimental Materials:

HUVSMC (Human Umbilical Vein Smooth Muscle Cells), FGFR2-Fcc, and COS-7 cells.

Experimental Methods:

Surface plasmon resonance (SPR) assay: FGFR2-Fc was fixed on a sensor, FGF2 or VEGF-B₁₆₇ was then added to analyze their binding to FGFR2.

Pull-down assay: 0.5 μg human FGFR2-Fc was added to 20 μl agarose beads combined with protein G for an overnight incubation at 4° C. After wash, VEGF-B or FGF2 (as positive control) of different doses were added for a 3-hour incubation at 37° C. SDS-PAGE was performed after the beads were washed by PBS, as to detect protein expression.

Alkaline phosphatase assay of FGFR2: the COS-7 cells were transfected with FGFR2-AP (alkaline phosphatase) expressing plasmid, and were further added with BSA, FGF2 (as positive control), VEGF-B₁₆₇ or PlGF1 for stimulation. Supernatant of cell culture medium was collected 3 days later to perform an activity assay of alkaline phosphatase.

Dot-blot assay: Human VEGF-B₁₆₇ or FGF2 protein (as positive control) of different doses (4.7, 19, 75, 300 and 1200 ng) were respectively dotted on a upper row and a middle row of a film, FGFR2c-Fc protein of different doses (1.2, 4.7, 19, 75 and 300 ng) and were dotted on a lower row.1 μg/ml FGFR2c-Fc was added to the film blocked by BSA for incubation, the film was further incubated by peroxidase-labeled human IgG Fcγ to color.

In situ proximity ligation assay: the operation was performed according to the instruction of Duolink II PLA kit (Sigma, DUO92007). HUVSMC was stimulated with BSA, VEGF-B₁₆₇ or PlGF. The operational procedure of the PLA assay followed that of Embodiment 5 (except that anti-FGFR2 antibody was used).

Dynamic binding assay: A dynamic binding assay was performed between VEGF-B₁₆₇ and FGFR2. The OD (optical density) of the binding VEGF-B₁₆₇ was measured as the concentration of VEGF-B₁₆₇ increased (in the form of different concentration group).

Experimental Results:

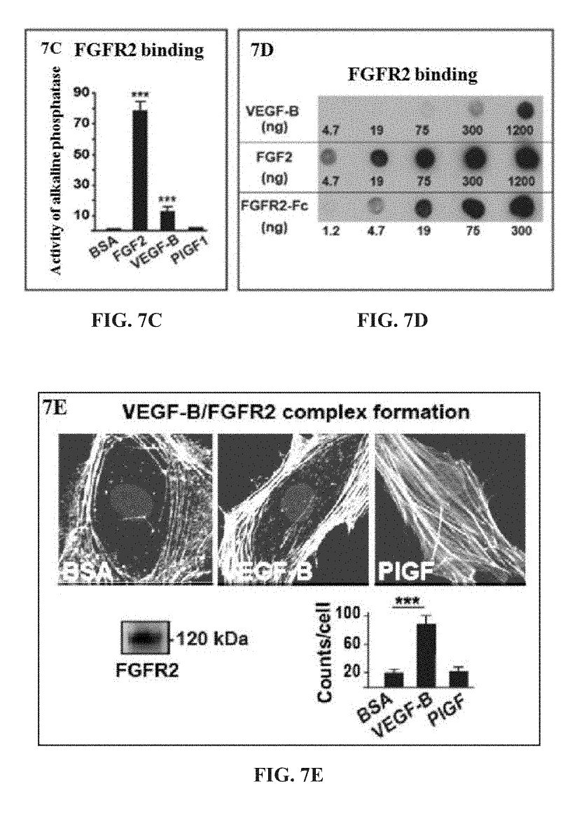

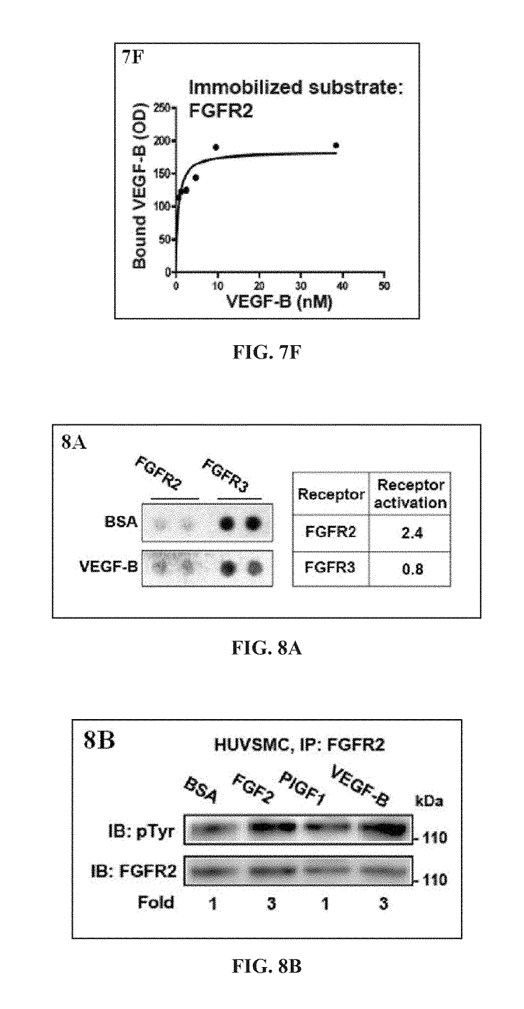

As illustrated in FIG. 7A-7F, the result of the SPR assay (FIG. 7A) shows that VEGF-B₁₆₇ bound to FGFR2 with a Kd value of 112 nM.

Pull-down assay (FIG. 7B) also indicates that VEGF-B₁₆₇ binds to FGFR2.

The result of the alkaline phosphatase assay of FGFR2 (FIG. 7C) confirms that VEGF-B₁₆₇ binds to FGFR2, while PlGF1 has no such function.

The result of the dot-blot assay (FIG. 7D) also shows that VEGF-B₁₆₇ binds to FGFR2.

The result of the in situ proximity ligation assay (FIG. 7E) shows that the exogenous VEGF-B₁₆₇ induced the formation of a VEGF-B₁₆₇/FGFR2 complex, while PlGF1 had no such function.

The result of the dynamic binding assay (FIG. 7F) also indicates that VEGF-B₁₆₇ binds to FGFR2.

### Embodiment 8: VEGF-B₁₆₇ Induces the Phosphorylation of FGFR2

Experimental Materials:

HUVSMC, HREC, HMVEC, PAE-FGFR2c, PC3 and OVCAR4.

Experimental Methods:

Co-immunoprecipitation and antibody chip assay for phosphorylation: HUVSMC were stimulated with BSA, FGF2, PlGF1 or VEGF-B₁₆₇. HREC were stimulated with FGF2 or VEGF-B₁₆₇ for different time lengths (0, 10, 30, 60 and 120 mins). HMVEC, PAE-FGFR2c, PC3 and OVCAR4 were stimulated with BSA, FGF2 or VEGF-B₁₆₇. Total protein was extracted respectively from the different cells and then incubated with FGFR2 antibody for co-immunoprecipitation (with pTyr), the phosphorylation level of FGFR2 was detected by performing an RTK (Receptor Tyrosine Kinase) antibody array.

Experimental Results:

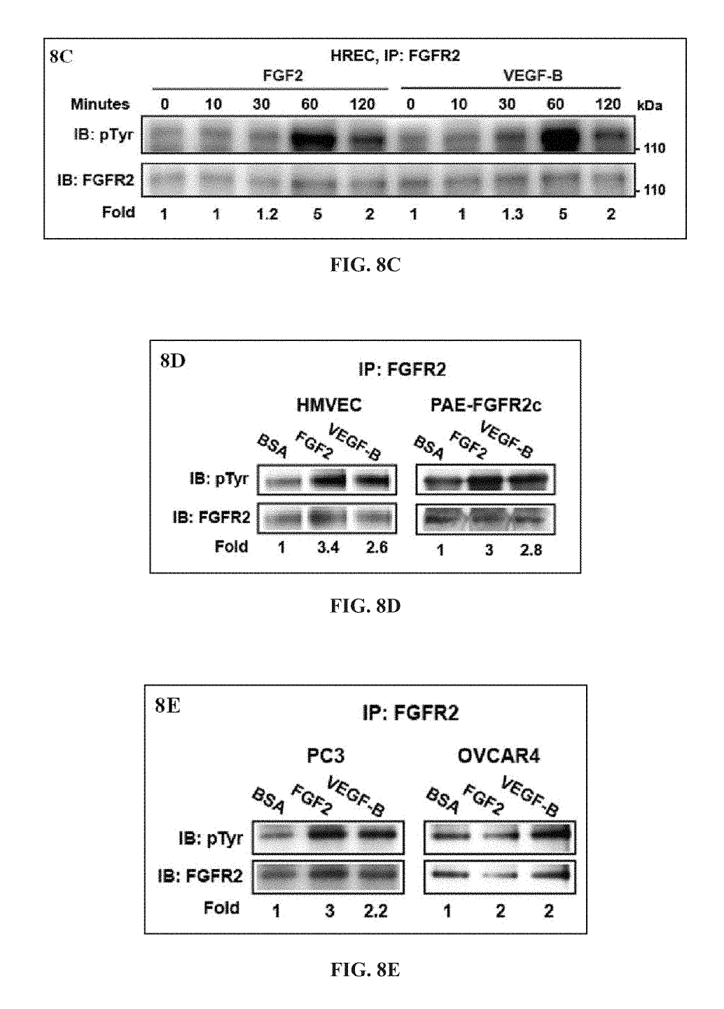

As illustrated in FIG. 8A-8E, the result of the antibody chip assay for phosphorylation (FIG. 8A) shows that VEGF-B₁₆₇ induced the phosphorylation of FGFR2, while the phosphorylation level of FGFR3 remained unaffected.

The result of the co-immunoprecipitation shows that in HUVSMC (FIG. 8B) and HREC (FIG. 8C), VEGF-B₁₆₇ roughly equaled to FGF2 in respect of inducing the phosphorylation of FGFR2, while PlGF had no such significant inducement.

The result of the co-immunoprecipitation (FIG. 8D-8E) also shows that VEGF-B₁₆₇ roughly equaled to FGF2 in respect of inducing the phosphorylation of FGFR2 in different cells including HUVEC, PAE-FGFR2c, PC3 and OVCAR4.

### Embodiment 9: VEGF-B₁₆₇ Induces Interaction Between FGFR2 and VEGFR1

Experimental Materials:

8-week-old C57B16 mice and mouse primary smooth muscle cells (SMC).

Experimental Methods:

Extraction of the mouse primary smooth muscle cells: Aortas of the 8-week-old C57B16 mice were separated and added to a digestive solution containing 175 U/ml collagenase and 1.25 U/ml elastase for a 25-minute incubation at 37° C. The aortic adventitia was removed under a stereoscope. The obtained smooth aortas were transferred to DMEM medium containing 10% FBS and cultured overnight in an incubator at 37° C. On the second day, the aortas were added to another digestive solution containing 175 U/ml collagenase and 2.5 U/ml elastase for a 60-minute incubation at 37° C. The vascular tissue was gently disassociated into 1 mm pieces and seeded in a culture dish for continuing culture.

Co-Immunoprecipitation: brain tissue and retinae were extracted from the C57B16 mice. The C57B16 mice were intravitreally injected with BSA (500 ng/eye), FGF2 (100 ng/eye) or VEGF-B₁₆₇ (500 ng/eye), and their retinae were extracted 1 hour after the injection. The brain tissue and retinae, and the retinae from the injected C57B16 mice were treated by the operational procedure described in Embodiment 5, and incubated with anti-FGFR2 antibody for co-immunoprecipitation (with VEGFR1 or VEGFR2).

In situ proximity ligation assay (PLA): mouse primary SMC were stimulated with BSA, VEGF-B₁₆₇ or PlGF1. The operational procedure of the PLA followed that of Embodiment 5 (except that anti-FGFR2 antibody was used).

Experimental Results:

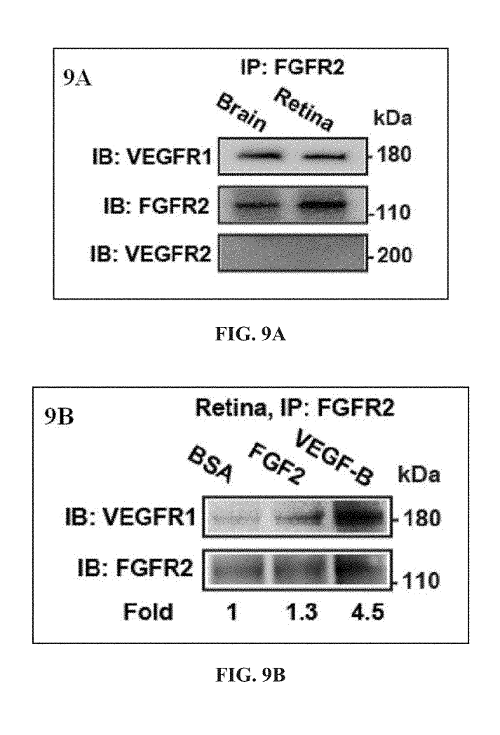

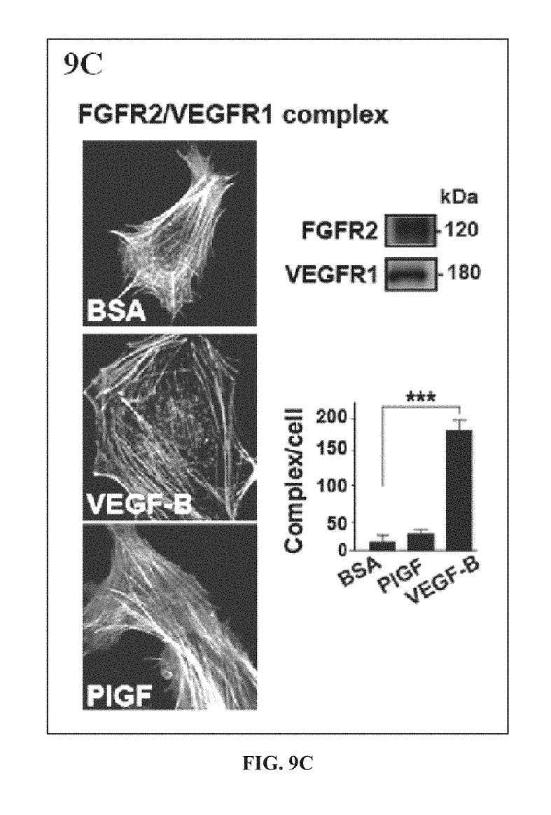

As illustrated in FIG. 9A-9C, the result of the co-immunoprecipitation assay (FIG. 9A) indicates that in brain and retinal tissues, FGFR2 binds to VEGFR1 but not to VEGFR2.

It is indicated that VEGF-B₁₆₇ promoted the interaction between FGFR2 and VEGFR1 after the injection of VEGF-B₁₆₇ into the vitreous bodies of the mice (FIG. 9B).

The result of the in situ proximity ligation assay (FIG. 9C) shows that the exogenous VEGF-B₁₆₇ induced the formation of a FGFR2/VEGFR1 complex, while PlGF had no such inducement.

### Embodiment 10: VEGF-B₁₆₇ Up-Regulates Spry4 Expression

Experimental Materials:

HUVSMC, EAhy926, OVCAR4 (human ovarian cancer cells), and mouse primary smooth muscle cells Fgfr2^{flox/flox} and Flt1^{flox/flox}.

Experimental Methods:

A fluorescence quantitative real-time PCR of Spry4 expression was performed on HUVSMC stimulated with VEGF-B₁₆₇ for different time lengths (0, 10 mins, 30 mins, 1 hr, 2 hrs and 6 hrs).

An immunoblot assay of Spry4 expression level was performed on the said cells which were first treated as follows:

HUVSMC were stimulated with BSA or VEGF-B₁₆₇;

Flt1^{flox/flox} SMC (having Flt1^{flox/flox} knocked out by Ad-Cre or not) were stimulated with BSA or VEGF-B₁₆₇;

Fgfr2^{flox/flox} SMC (having Fgfr2^{flox/flox} knocked out by Ad-Cre or not) were stimulated with BSA or VEGF-B₁₆₇;

HUVSMC were stimulated with BSA, FGF2 or VEGF-B₁₆₇;

Endothelial cells EAhy926 were stimulated with BSA or VEGF-B₁₆₇ for 24 and 48 hours;

OVCAR4 were stimulated with BSA, or with VEGF-B₁₆₇ for 6, 12, 20, 30 and 40 hours.

Steps of the fluorescence quantitative real-time PCR, immunoblot assay and gene knockout refer to the aforesaid embodiments.

Experimental Results:

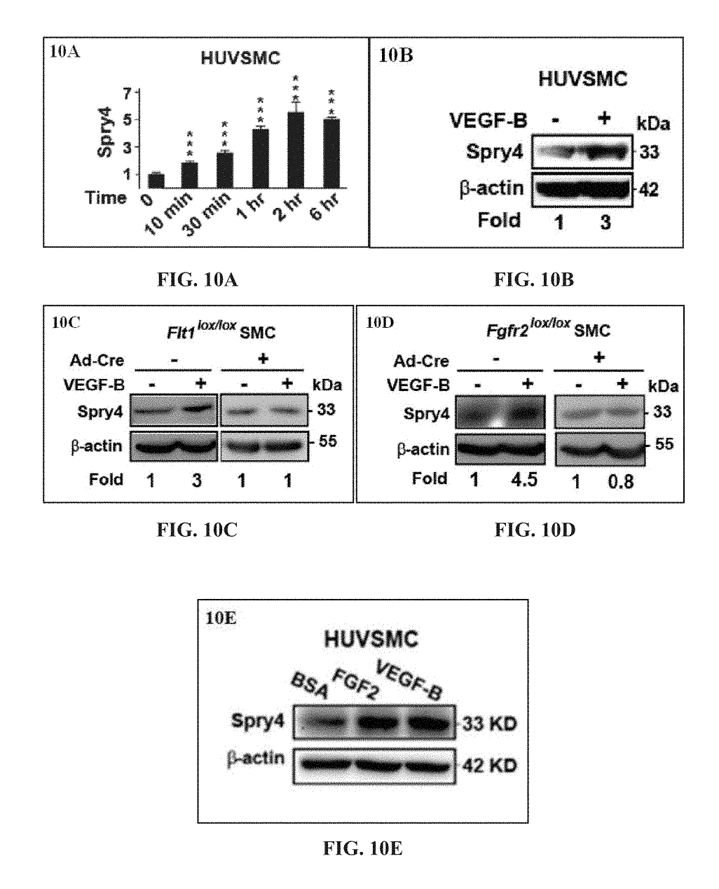

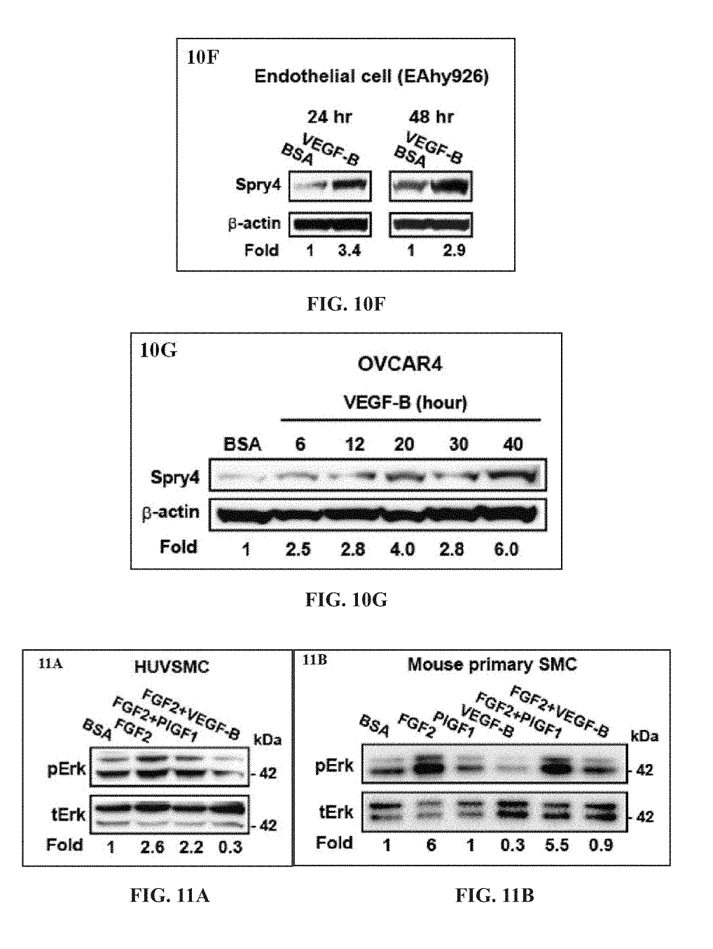

As illustrated in FIG. 10A, it is shown that in mRNA level, Spry4 expression was up-regulated in HUVSMC stimulated with VEGF-B₁₆₇.

As illustrated in FIG. 10B, it is shown that in protein level, Spry4 expression was up-regulated in HUVSMC stimulated with VEGF-B₁₆₇.

As illustrated in FIG. 10C, it is shown that VEGF-B₁₆₇ up-regulated Spry4 expression in the presence of VEGFR1, while VEGF-B₁₆₇ could not up-regulate Spry4 expression when VEGFR1 was knocked-down.

As illustrated in FIG. 10D, it is shown that VEGF-B₁₆₇ up-regulated Spry4 expression in the presence of FGFR2, while VEGF-B₁₆₇ could not up-regulate Spry4 expression when FGFR2 was knocked down.

The result of the immunoblot assay shows that VEGF-B₁₆₇ acted on HUVSMC (FIG. 10E), endothelial cells EA.Hy926 (FIG. 10F) and ovarian cancer cells OVCAR4 (FIG. 10G) as up-regulating Spry4 expression.

### Embodiment 11: VEGF-B₁₆₇ Inhibits FGF2 from Phosphorylating Erk Via VEGFR1, FGFR2 and Spry4

Experimental Materials:

HUVSMC, PAE (Porcine Aortic Endothelial cells), and mouse primary smooth muscle cells (SMC) Fgfr2^{flox/flox}, Flt1^{flox/flox}, Spry4^{-/-} and Flt1-tk^{-/-}.

Experimental Methods:

These different cells were stimulated with FGF2 or VEGF-B₁₆₇, and an immunoblot assay was performed to analyze the effect of such stimulations on phosphorylation of Erk. The steps of the immunoblot assay refer to the aforesaid embodiments. These different cells were treated as follows:

HUVSMC were stimulated with BSA, FGF2, FGF2+PlGF1 or FGF2+VEGF-B₁₆₇;

Mouse primary SMC were stimulated with BSA, FGF2, PlGF1, VEGF-B₁₆₇, FGF2+PlGF1 or FGF2+VEGF-B₁₆₇;

Flt1^{flox/flox} SMC (having Flt1^{flox/flox} knocked out by Ad-Cre or not) were stimulated with BSA, FGF2, PlGF1, VEGF-B₁₆₇, FGF2+PlGF1 or FGF2+VEGF-B₁₆₇;

Flt1-tk^{+/+} (wild type) SMC and Flt1-tk^{-/-} SMC were stimulated with BSA, FGF2, PlGF1, VEGF-B₁₆₇, FGF2+PlGF1 or FGF2+VEGF-B₁₆₇;

Fgfr2^{flox/flox} SMC (having Fgfr2^{flox/flox} knocked out by Ad-Cre or not) were stimulated with BSA, FGF2, PlGF1, VEGF-B₁₆₇, FGF2+PlGF1 or FGF2+VEGF-B₁₆₇;

Spry4^{+/+} (wild type) SMC and Spry4^{-/-} SMC were stimulated with BSA, FGF2, PlGF1, VEGF-B₁₆₇, FGF2+PlGF1 or FGF2+VEGF-B₁₆₇;

PAE-FGFR2 were stimulated with BSA, FGF2, PlGF1, VEGF-B₁₆₇. FGF2+PlGF1 or FGF2+VEGF-B₁₆₇.

Experimental Results:

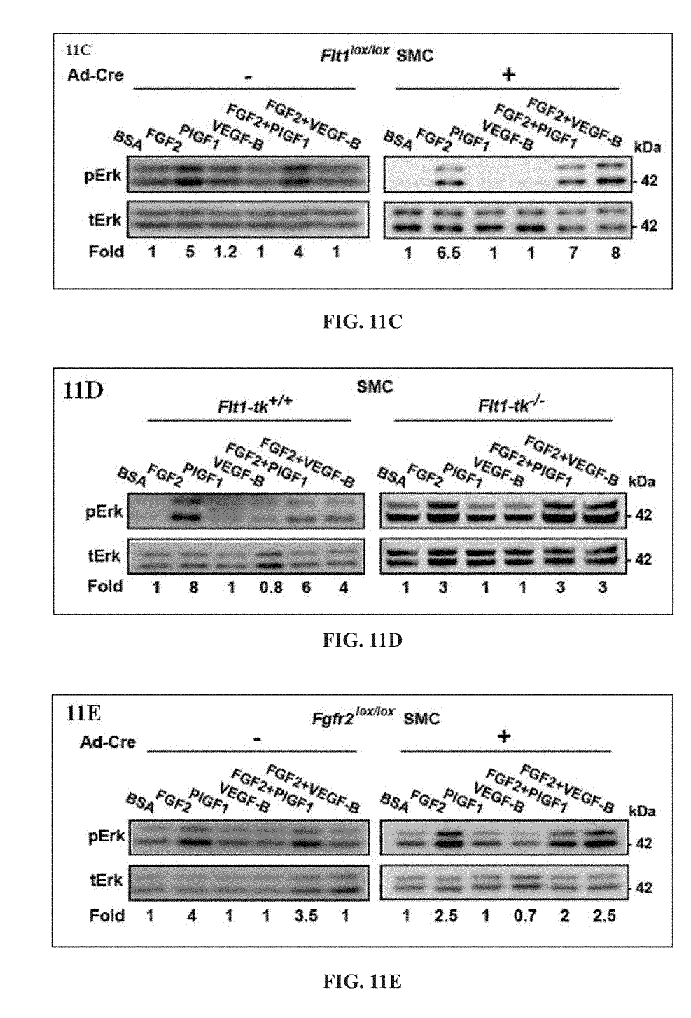

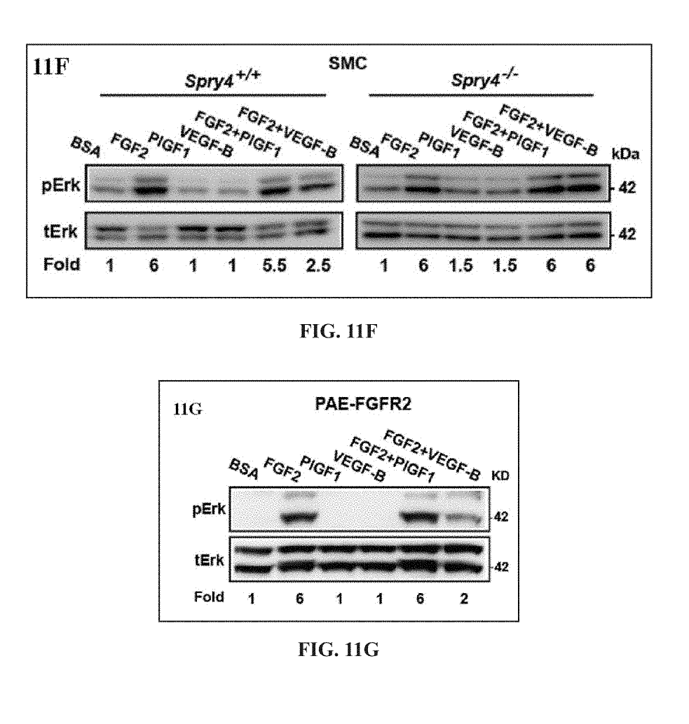

As illustrated in FIG. 11A-11G, it is shown that FGF2 induced the phosphorylation of Erk in the HUVSMC (FIG. 11A) and mouse primary SMC (smooth muscle cells, FIG. 11B), while such inducement was weakened by the addition of VEGF-B₁₆₇. PlGF1 had no such inducement.

As illustrated in FIG. 11C, it is shown that in the vascular smooth muscle cells (SMC) separated from the Flt1^{flox/flox} mice, VEGF-B₁₆₇ inhibited the FGF2-induced phosphorylation of Erk in the presence of VEGFR1, while PlGF1 caused no such inhibition. VEGF-B₁₆₇ could not inhibit the FGF2-induced phosphorylation of Erk when VEGFR1 was knocked out by the added Ad-cre.

As illustrated in FIG. 11D, it is shown that VEGF-B₁₆₇ inhibited the FGF2-induced phosphorylation of Erk in the presence of VEGF-B₁₆₇ tyrosine kinase, while PlGF1 had no such inhibition. Such inhibition disappeared when VEGFR1 tyrosine kinase gene was knocked out.

As illustrated in FIG. 11E, it is shown that VEGF-B₁₆₇ inhibited the FGF2-induced phosphorylation of Erk in the presence of FGFR2, while PlGF1 caused no such inhibition. Such inhibition disappeared when FGFR2 is knocked down.

As illustrated in FIG. 11F, it is shown that VEGF-B₁₆₇ inhibited the FGF2-induced phosphorylation of Erk in the presence of Spry4, while PlGF1 caused no such inhibition. Such inhibition disappeared when Spry4 was knocked out.

As illustrated in FIG. 11G, it is shown that in the PAE cells overexpressing FGFR2 (PAE-FGFR2), VEGF-B₁₆₇ inhibited FGF2 from activating Erk, while PlGF1 had no such inhibition. This indicates that VEGF-B₁₆₇ specifically inhibited FGF2 from activating Erk.

### Embodiment 12: VEGF-B₁₆₇ Inhibits the Effects of FGF2 on Vascular Smooth Muscle Cells and Neoangiogenesis

Experimental Materials:

HUVSMC and VEGF-B₁₆₇^{-/-} mice.

Experimental Methods:

Cell proliferation assay: the HUVSMC were plated in a 96-well plate with 2,000 cells in each well, and were starved overnight in serum-free DMEM medium. On the second day, the wells were added with BSA, FGF2, VEGF-B or other factors. The cells were cultured in an incubator at 37° C. with 5% CO₂ for 48 hours, and then each well was added with 20 μl MTT solution. Supernatant of each well was extracted 4 hours later, and then each well was added with 150 μl DMSO to dissolve precipitate. Absorbance at 570 nm wavelength was measured.

Cell migration assay: HUVSMC cells were plated in a 6-well plate for a 100% confluence. Manually scraped the cell monolayer with a 200 μl pipette tip for creating wounds and acquired images. The cells were added with FGF2, VEGF-B₁₆₇ or other stimulants, and were imaged 24 hours later. The number of the migrating cells was counted.

Immunostaining assay: retinae from C57B16 mice (wild type) and VEGF-B₁₆₇^{-/-} mice were extracted, sectioned and immunostained by NG2+IB4+DAPI, and the rate of NG2-positive field under microscope was measured. The steps of the immunostaining assay refer to the aforesaid embodiments.

Experimental Results:

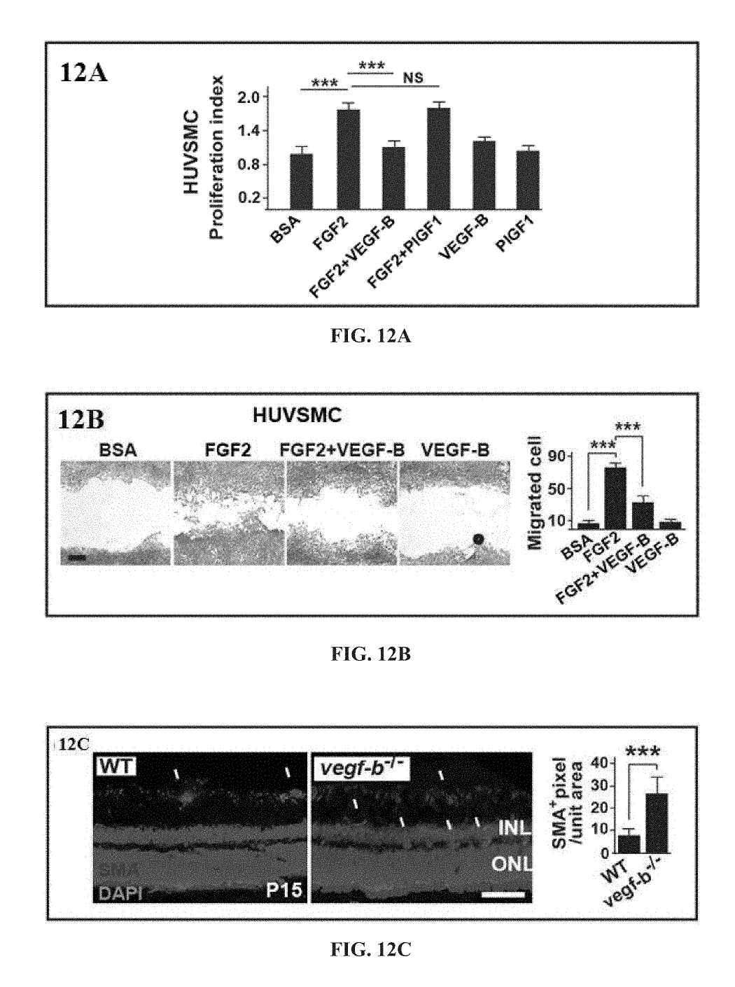

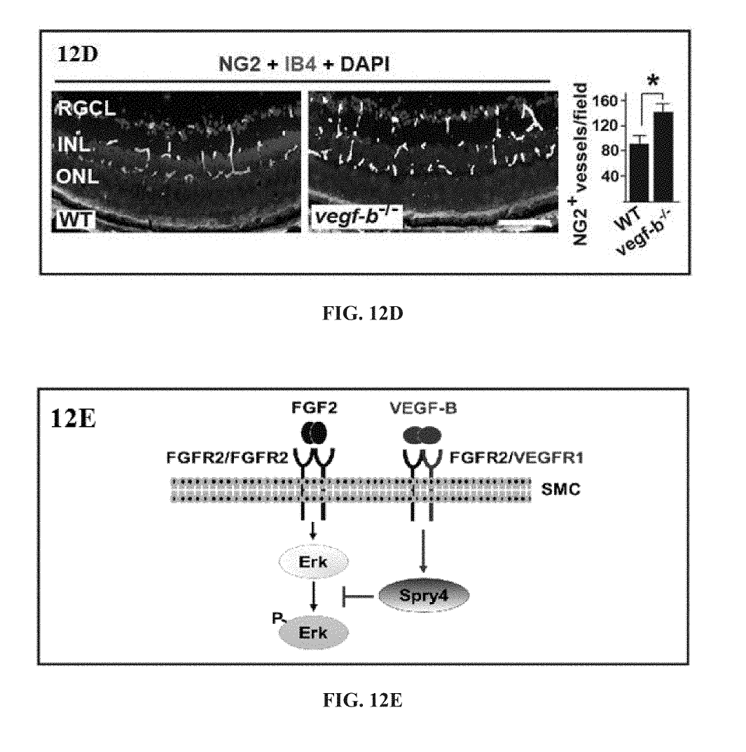

As illustrated in FIG. 12A-12E, the result of the cell proliferation assay (FIG. 12A) shows that VEGF-B₁₆₇ inhibited the FGF2-induced HUVSMC proliferation, while PlGF1 caused no such inhibition.

The result of the cell migration assay (FIG. 12B) shows that VEGF-B₁₆₇ inhibited the FGF2-induced cell migration.

The result of the retina sectioning and staining (FIG. 12C) shows increases in both marker protein expression in the retinal vascular smooth muscle cells and retinal vascular density in the VEGF-B₁₆₇ gene deficient mice.

The result of the retina sectioning and staining (FIG. 12D) also shows an increase in the ratio of pericytes in retinal blood vessels in the VEGF-B₁₆₇ gene deficient mice.

As illustrated in FIG. 12E, it is indicated that VEGF-B₁₆₇ induces the formation of FGFR2/VEGFR1 complex, upregulates Spry4 expression, and inhibits Erk activation and FGF2 signaling pathway.

It should be noted that, the embodiments disclosed above are only used to illustrate the technical scheme of the invention, not to limit the scope of the invention. Despite that the illustration is made in reference to the preferred embodiments, those skilled in the art should understand that many improvements and alternatives can be made without departing from the principle of the invention, these improvements and alternatives should also be included in the scope of the invention.

1. A method of treating a disease involving neoangiogenesis in a patient, comprising:
administering VEGF-B to the subject.

2. The method according to claim 1, wherein the disease involving neoangiogenesis is a proliferative disease.

3. The method according to claim 2, wherein the proliferative disease is a cancer.

4. The method according to claim 3, wherein the cancer is selected from the group consisting of liver cancer, endometrial cancer, breast cancer, bladder cancer, rectal cancer, cervical cancer, ovarian cancer and melanoma.

5. The method according to claim 1, wherein the VEGF-B treats the disease via inhibiting the neoangiogenesis.

6. The method according to claim 5, wherein the VEGF-B inhibits the neoangiogenesis by inhibiting an FGF2-induced phosphorylation of Erk.

7. The method according to claim 6, wherein the VEGF-B inhibits the FGF2-induced phosphorylation of Erk by competing with FGF2 for binding to FGFR1 and/or FGFR2.

8. The method according to claim 6, wherein the VEGF-B inhibits the FGF2-induced phosphorylation of Erk by up-regulating Spry4 expression.

9. The method according to claim 8, wherein the VEGF-B up-regulates the Spry4 expression by inducing the formation of an FGFR1/VEGFR1 complex and/or an FGFR2/VEGFR1 complex.

10. The method according to claim 1, wherein the VEGF-B is in the form of VEGF-B protein, VEGF-B expressing plasmids, VEGF-B expressing viruses and/or VEGF-B expressing cells.

11. The method according to claim 1, wherein the VEGF-B is VEGF-B₁₆₇ and/or VEGF-B₁₈₆.

12. The method according to claim 1, wherein the VEGF-B is a modified VEGF-B, the modified VEGF-B is a cyclized, phosphorylated and/or methylated VEGF-B; or the VEGF-B is a recombinant protein or polypeptide having 1-5 more or less amino acids than the VEGF-B.

13. The method according to claim 1, further comprising:
administering an inhibitor of FGF2 receptor to the subject.

14. The method according to claim 13, wherein the FGF2 receptor is FGFR1 and/or FGFR2.

15. A pharmaceutical composition comprising VEGF-B protein, VEGF-B expressing plasmids, VEGF-B expressing viruses and/or VEGF-B expressing cells as active ingredients for treating a disease involving neoangiogenesis.

16. The pharmaceutical composition according to claim 15, further comprising an inhibitor of FGF2 receptor.

17. The pharmaceutical composition according to claim 16, wherein the FGF2 receptor is FGFR1 and/or FGFR2.
